(19) United States
(12) Patent Application Publication
(10) Pub. No.: US 2011/0305765 A1
(43) Pub. Date: Dec. 15, 2011

(54) PREPARATION AND METHODOLOGY OF SILK FIBROIN NANOPARTICLES

(75) Inventors: Anshu B. Mathur, Missouri City, TX (US); Carmen N. Rios, Port Arthur, TX (US); Vishal Gupta, Houston, TX (US); Abraham Aseh, Waco, TX (US)

(73) Assignee: BOARD OF REGENTS, THE UNIVERSITY OF TEXAS SYSTEM, Austin, TX (US)

(21) Appl. No.: 13/129,370

(22) PCT Filed: Nov. 20, 2009
(86) PCT No.: PCT/US09/65374

§ 371 Date: Sep. 2, 2011

Related U.S. Application Data
(60) Provisional application No. 61/116,945, filed on Nov. 21, 2008.

(51) Int. Cl. A61K 9/14; A23L 1/304; A23L 1/302; A23L 1/303; A23L 1/30; A61K 38/02; A61K 31/7088; A61K 31/12; A61K 31/122; A61P 35/00; A61P 29/00; A61P 23/00; A61P 31/00; A23L 1/305; A61K 39/395; B82Y 5/00

(52) U.S. Cl. 424/491; 424/400; 424/130.1; 426/2; 426/72; 426/73; 426/648; 427/2.14; 514/1.1; 514/44 R; 514/679; 514/680; 977/773; 977/795; 977/906

(57) ABSTRACT

Disclosed are nanoparticles for delivery of drugs and/or nutraceuticals that include a fibroin polypeptide and a drug or nutraceutical, wherein the nanoparticle has a diameter of about 1 nm to about 500 nm, and compositions of the nanoparticles. The nanoparticles may further include a chitosan, or a proteoglycan such as decorin. Also disclosed are methods of delivering a drug and/or nutraceutical to a subject that involve administering to the subject nanoparticles of the present invention. Also disclosed are methods of making the nanoparticles of the invention, and kits that include the nanoparticles of the invention.

This application claims the benefit of the filing date of U.S. Provisional Patent Application Ser. No. 61/116,945, filed Nov. 21, 2008, the entire contents of which is hereby incorporated by reference.

### BACKGROUND OF THE INVENTION

1. Field of the Invention

The present invention relates generally to the fields of drug delivery, nutraceuticals, nanoparticles, molecular biology, and therapeutics. More particularly, the invention concerns fibroin nanoparticles, such as silk fibroin nanoparticles, for the delivery of therapeutic agents, diagnostic agents, or nutraceuticals into cells and tissues, and compositions, methods, and kits that involve the fibroin nanoparticles of the present invention. The invention also concerns nanoparticles that include a blend of silk fibroin and one or more additional agents, such as a chitosan or a proteoglycan such as decorin.

2. Description of Related Art

Cancer is one of the most common causes of morbidity and mortality in the world. Unfortunately, treatment of cancer using pharmaceutical agents is often limited by reduced bioavailability, lack of tissue specificity, and tissue toxicity of the therapeutic agent. For example, curcumin is a yellow polyphenol extracted from the rhizome of turmeric, which has shown good activity as an anti-cancer agent by inhibiting proliferation of various cancers (Kunnumakkara et al., 2008). However, the treatment of solid tumors such as pancreatic cancer, cervical cancer, and breast cancer with chemotherapeutic agents such as curcumin is limited by the lack of bioavailability and tissue specificity (Anand et al., 2007).

To enhance the bioavailability of curcumin, several approaches have been taken including curcumin nanoparticles. The use of copolymers that are biodegradable, biocompatible, and non-toxic has been employed in the formulation of drugs. Recent studies demonstrated various formulations of curcumin nanoparticles using polymeric materials (Sahu et al., 2008; Bisht et al., 2007), solid lipids (Tiyaboonchai et al., 2007), and liposomes (Sou et al., 2008; Li et al., 2005; Kunwar et al., 2006). However, none of the above formulations were derived from natural polymers to eliminate tissue toxicity completely. Although the use of liposomes reduced the toxicity, there is no tissue specificity associated with them.

Curcumin has been shown to suppress many tumerogenic pathways including Her2 pathway in breast cancer cells (Kunnumakkara et al., 2008; Hong et al., 1999). However, none of the nanoparticle studies demonstrated the efficacy of nanocurcumin on breast cancer cells or tumors.

Silk fibroin (SF) is a protein created by Bombyx mori (silkworms) in the production of silk. Silk consists of two main proteins—sericin and SF. SF consists of layers of antiparallel beta sheets that contribute to the tensile strength of silk. SF is also highly elastic. The removal of sericin coating from silk ensures non-inflammatory or non-toxic response (Santin et al., 1999). Also, the SF show strong affinity towards polysaccharides and possess high strength and flexibility (Roden et al., 1985; Altman et al., 2003). SF coating has been shown to increase retention of emodin in breast cancer cells thereby increasing the efficacy of therapeutic (Cheema et al., 2007).

Silk microspheres (Wang et al., 2007; Wenk et al., 2008), nanolayers (Wang et al., 2007) and coatings on drug-loaded liposomes (Gobin et al., 2006) have been used previously for controlled release. The coating of SF on drug-loaded liposomes increases the adhesion and residence time, and brings the drug in close proximity to the cell (Cheema et al., 2007; Gobin et al., 2006). Loading the drug in liposomes and then coating with SF is a two-step process, which makes the particle size larger than 100 nm (Gobin et al., 2006).

Chitosans have been evaluated as drug carriers in nanoparticles. A chitosan (CS) is a cationic polysaccharide derived from chitin, which is a copolymer of glucosamine and N-acetyl glucosamine units (Mi et al., 1999; Gupta and Ravi Kumar, 2001). CS contains high charge density, non toxic, sequester growth factors and have been shown to have wound healing properties (VandeVord et al., 2002). Chitosans have been evaluated as carriers for drugs in view of their biocompatilibity and biodegradability (Bayomi et al., 1998; Genta et al., 1998; Ko et al., 2003; Katas and Alpar, 2006). There have been limited reports concerning nanoparticles that include chitosan and TPP for delivery of siRNA (Katas and Alpar, 2006; Liu et al., 2007). Scaffolds that include a blend of SF and CS have been previously described for musculofascial regeneration in ventral hernia repair (Gobin et al., 2006).

Thus, there is the need for alternative drug delivery formulations to enhance drug bioavailability, target specific cells, and minimize risk of tissue toxicity while optimizing therapeutic outcomes.

### SUMMARY OF THE INVENTION

The present invention is in part based on the finding that incorporation of a drug and/or nutraceutical into a nanoparticle that includes a fibroin results in enhanced bioavailability and improved efficacy of the drug and/or neutraceutical. Further, there may be a reduction in tissue toxicity. Further, the nanoparticles possess excellent cell adhesion characteristics with a low likelihood of inflammatory and thrombogenic responses. For example, the inventors have prepared silk fibroin (SF) nanoparticles (with or without chitosan) that include curcumin and the HIV drug R15K peptide and have found that these nanoparticles provide more enhanced delivery of therapeutic agent to diseased cells. The method of targeting cells, such as diseased cells, is related to the ability of SF to self-assemble into beta sheet structures. As a corollary, the beta sheet structure of amyloid proteins of the prions has been shown to be the main driving force for the infection of cells in mad cow disease, as these structures are able to cross the blood-brain barrier.

The present invention generally provides for nanoparticles or complexes for delivery of a drug and/or nutraceutical. In some embodiments, the nanoparticle includes (a) a fibroin polypeptide and (b) a drug and/or nutraceutical, wherein the nanoparticle or complex has a diameter of about 1 nm to about 500 nm. The fibroin may be any type of fibroin, but in particular embodiments it is silk fibroin polypeptide from the silkworm Bombyx mori (hereinafter “silk fibroin” abbreviated as SF; SEQ ID NO:1; GenBank Accession No. AAL83649). Other examples of fibroin include spider silk fibroin and other insect silk fibroins. Other examples of fibroins include fibroin from Antipaluria urichi (GenBank Accession No. ACJ04053; SEQ ID NO:2); fibroin from Oecophylla smaragdina (GenBank Accession No. ABW21705; SEQ ID NO:3); fibroin from Oecophylla smaragdina (GenBank Accession No. ABW21703; SEQ ID NO:4); fibroin from Mymecia forficata (GenBank Accession No. ABW21701; SEQ ID NO:5); and fibroin from Bombus terrestris (GenBank Accession No. ABW21697; SEQ ID NO:6). The fibroin may be genetically engineered, chemically synthesized, or obtained from natural sources. The fibroin may be produced from genetically engineered cells in vivo or in vitro.

A “polypeptide” as used herein refers to a consecutive series of 2 or more amino acids, and as used herein encompasses the terms “peptide” and “protein.” Therefore, in some embodiments, the polypeptide comprises a consecutive series of at least 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 224, 226, 228, 230, 232, 234, 236, 238, 240, 242, 244, 246, 248, 250, 252, 254, 256, 258, 260, 262, or the full-length amino acid sequence of a full-length fibroin protein, or any range of numbers of consecutive sequences of amino acids derivable herein. Thus, for example, a SF polypeptide may comprise between 10 and 262, between 20 and 250, between 30 and 220, between 40 and 200, between 50 and 180, or between 60 and 120 consecutive amino acids of SEQ ID NO:1. The fibroin polypeptide may include one or more additional amino acid residues at the C-terminus or N-terminus of the consecutive sequence of amino acids of the fibroin polypeptide. In some embodiments, the fibroin polypeptide has at least 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, 99% or greater sequence homology to a known fibroin protein, such as SF. The fibroin may or may not encapsulte the drug.

In some embodiments, the nanoparticle or complex has a diameter of about 1 nm to about 300 nm. In more specific embodiments, the drug delivery nanoparticle has a diameter of about 1 nm to about 100 nm.

The fibroin polypeptide may coat a core that includes the drug and/or nutraceutical. In other embodiments, the fibroin polypeptide and the drug/nutraceutical are admixed together to form nanoparticles without a defined core. The nanoparticles set forth herein may include a single drug/nutraceutical, or more than one drug/nutraceutical. A “drug” as used herein references to any therapeutic or diagnostic agent. Therapeutic agents include agents that can be applied in the treatment or prevention of a disease or health-related condition. Diagnostic agents include agents that can be applied in determining the presence or absence of a disease or health-related condition in a subject. The drug may be a small molecule, a peptide, a polypeptide, a protein, a lipid, a carbohydrate, an antibody, an antibody fragment, a DNA a RNA, or a combination thereof (such as a lipoprotein).

In particular embodiments, the drug is a therapeutic agent. Any therapeutic agent is contemplated for inclusion in the nanoparticles of the present invention. Non-limiting examples of types of therapeutic agents include an anticancer agent, an anti-inflammatory agent, an antimicrobial agent, an analgesic, a hormone, an agent that can be applied in the treatment of a degenerative disease, and an anesthetic agent. In particular embodiments, the therapeutic agent is an anticancer agent. Any agent that can find application in the treatment or prevention of cancer is contemplated as an anticancer agent. Non-limiting examples of anticancer agents include curcumin, emodin, thiotepa, cyclosphosphamide, busulfan, topotecan, chlorambucil, melphalan, carmustine, lomustine, actinomycin, bleomycin, dactinomycin, daunorubicin, mitomycin C, methotrexate, 5-fluorouracil (5-FU), 6-mercaptopurine, thioguanine, arabinoside, cyclophosphamide, thiotepa, paclitaxel, doxetaxel, chlorambucil, gemcitabine, 6-thioguanine, mercaptopurine, methotrexate, cisplatin, oxaliplatin, carboplatin, vinblastine, etoposide, vincristine, retinoic acid, cisplatin (CDDP), carboplatin, procarbazine, and mechlorethamine. In some specific embodiments, the drug is a curcuminoid. Non-limiting examples of curcuminoids include curcumin, demethoxycurcumin, and bisdemethoxycurcumin. The drug may be a derivative of a curcuminoid. For example, the drug may be a tetrahydrocurcuminoid. In further specific embodiments, the drug is emodin.

In further specific embodiments, the therapeutic agent is an agent that can be applied in the treatment of HIV infection. In certain specific embodiments, the therapeutic agent is I15K peptide.

The drug may also be an agent that can be applied in the diagnosis of disease (i.e., a diagnostic agent). For example, the diagnostic agent may be an agent that can be applied in imaging, such as CT, MRI, PET, SPECT, ultrasound, or other type of imaging. In some embodiments, the diagnostic agent is a quantum dot. For example, coating a quantum dot with fibroin as set forth herein may reduce toxicity of the quantum dot and facilitate tissue-targeting.

A “nutraceutical” as used herein includes any agent, food, or part of a food, that provides medical or health benefits to a subject. Included in this definition are dietary supplements. Non-limiting examples of nutraceuticals include vitamins, minerals, herbs or other bonanicals, amino acids, and so forth. They may aid in the treatment and/or prevention of a disease and/or disorder, or may aid in maintaining a state of health or well-being.

Nutraceuticals include, for example, vitamins and dietary supplements. Non-limiting examples of vitamin and dietary supplements include vitamin A, vitamin D, acetyl carnitine, vitamin B-12, vitamin B-3, vitamin B-6, C ester, calcium citrate, cholesterol, chromium, CLA (tonalin), cod liver oil, creatine, vitamin D, CHEA, Dong Quai, vitamin E, fish oil, gaba, vitamin A, acidophilus, alpha lipoic, vitamin B-1, vitamin B-150, vitamin B-5, vitamin B-9, biotin, vitamin C, calcium, chlorella, choline, coenzyme Q-10, cranberry extract, D-ribose, EDTA, flaxseed oil, acai, arginine, aspirin, vitamin B-100, vitamin B-2, B-complex, beta-carotene, brewers yeast, magnesium, chia, chlorophyll, chondroitin, glucosamine, DHA, DMAE, ester-C, fiber, folic acid, vitamin B-9, ginseng, gymneme sylvestre, hawthorn, hoodia, vitamin K, L-carnitine, hyaluronic acid, inositol, L-lysine, lecithin, lipoic, melatonin, glutamine, niacin, pantothenic acid, peppermint extract, phosphotidyl serine, potassium, policosanol, psyllium, pycnogenol, quercetin, resveratrol, retinol, shark cartilage, sambucus, selenium, silica, ubiquinol, valerian, vitamin B-15, chasteberry extract, wild yam extract, whey protein, yeast, and zinc.

Non-limiting examples of herbs include buta, aligator yam, alfalfa, almond, amalaki, ashwagandha, asoka tree, ambrette plant, apricot, arecanut palm, arjuna, aloe vera, arnica, ash gourd, ashoka tree, asparagus, babchi seed, bacopa, bael tree, bahama grass, banyan tree, barbados aloe, buddhist bauhinia, belliric myrobalan, bengal gram, bermuda grass, betelnut palm, bindweed, bishop's weed, billilotan, bitter melon, black nightshade, boerhavia, bitter gourd, black cumin, black nightshade, black-oil plant, black pepper, black plum, bonduc fruit, boswellia, bread wheat, butea gum tree, cinnamon, cajuput tree, calendula, cal mint, camphor, caper, cayenne, cal mint, chyavanprash, clove, country mallow, crowfoot, caraway, cinnamon, coconut extract, cohost, cardamom, carilla fruit, carum, cassia, castor, celery, centella, ceylon cinnamon, chebulic myrobalan, chickpea, chicory, chilli, chiretta, chir pine, citron, climbing staff tree, clove, coconut, coffee-senna, common mallow, common olive, common sesban, common wheat, coneru, connessi bark, coomb teak, coriander, corn poppy, costus, country mallow, cow-itch plant, cowhage, crab apple, crowfoot, cucumber, cultivated apple, cultivated carrot, cumin, curacao aloe, cutch tree, date, dead sea apple, dandelion extract, deodar, dhub grass, downy oak, drumstick, didymocarpus, evening primrose oil, East Indian globe thistle, elderberry extract, Egyptian rattle pod, elephant creeper, emblic myrogalan, Englist garden marigold, eucalyptus, false black pepper, false pepper, fennel, fenugreek, fenugreek, fire flame bush, five-leaved chaste tree, elderberry extract, foetid cassia, gall nut, galls, garcinia, garden rue, garlic, giant potato, giant reed, gingelly, ginger, golden dock, golden shower, gotu kola, grape, greater galangal, gulancha tinospora, guduchi, guggul, gymnema, haritaki, henbane, Himalayan cedar, Himalayan silver birch, holy basil, horsegram, horse radish, hydrophilia, hyssop, aloe, berberry, bdellium, beech, cotton plant, dill, gooseberry, gum-arabic tree, jojoba, kelp, krill oil, jeevanti, king bitters, kino tree, laburnum, lilac, long pepper, madder, malabar nut, maca, nattokinase, goji, mango, morning glory, mucuna, musk mallow, musk root, nard, neem, nut grass, nutmeg, paper birch, pennywort, pasanavheda, pellitory, pitasara, pomegranate, prickly chaff flower, sarsaparilla, spikenard, sweet fennel, valerian, winter green, ink nut, intellect tree, ironwood tree, jafarabad aloe, jaman, jambolan, java plum, khas-khas, khus-khus, land caltrops, cardamum, lemon, lentil, lettuce, licorice, liquorice, lodh tree, loose skinned orange, loose jackot orange, lovage, malabar nut, mango, mangosteen, morning glory, grape seed extract, grape extract, green tea extract, lavender, lutein, milk thistle, nettle, mucuna, musk mallow, musk root, neti pot, noni, neem, negro coffee, nut grass, nutmeg, orchid tree, pasanavheda, pellitory, pitasara, papaya, peppermint, Persian rose, pistachio, pinang palm, pomegranate, pongam oil tree, pot-marigold, prickly-chaff flower, puncture vine, purging cassia, purple fleabane, rauwolfia, red pepper, red poppy, rough chaff tree, rohida tree, rosemary, rubbish cassia, sacred basil, saffron, salep orchid, sal tree, sandalwood tree, sarala, saxifraga ligulata, shavatari, shilajeet, shilapushpa, skis, small caltrops, spreading hogweed, sedge, sedom apple, sensitive plant, sesame, shoe-flower, shrivasa, silk cotton tree, small caltrops, small fennel, snake wood, soap nut, soapnut-tree of North India, sodom apple, Spanish jasmine, stevia, saw palmetto extract, spirulina, tea extract, turmeric, St. John's wort, Spanish Pellitory, spearmint, spreading hogweed, stinking weed, stone flower, strychnous tree, succory, sunflower, swallow-wort, sweet flag, sweet indrajao, sweet root, sweet violet, tamarisk, tankana, Tasmanian blue gum tree, tea tree thistle extract, tonalin, tellicherry bark, teri pods, caper bush, the creat, thorn apple, three leaved caper, thyme-leaved gratiola, tinospora gulancha, tomato, triphala, toothache tree, touch me not, tree turmeric, umbrellas edge, velvet bean vibhitake, wild asparagus, yarrow, and yohimbe.

The nanoparticles may also include additional ingredients other than drug/nutraceutical and fibroin. In certain specific embodiments, the particle further comprises a chitosan. Chitosans, and examples of chitosans, are discussed in the specification below. In particular embodiments, the chitosan has a deacetylation degree of greater than about 70%. In further embodiments, the chitosan has a deacetylation degree of greater than about 80%. In some embodiments, the chitosan has a deacetylation degree of between 70% and 99%.

The weight ratio of the SF polypeptide to the chitosan in the nanoparticle may be any ratio. In particular embodiments, the weight ratio of SF polypeptide to chitosan in the nanoparticle is about 1 to about 6. In more specific embodiments, the weight ratio of SF to chitosan is about 2 to about 4.

In still further embodiments the nanoparticles include a fibroin and a proteoglycan. In particular embodiments the proteoglycan is decorin.

The present invention also concerns compositions that include the a drug delivery nanoparticle of the present invention and a carrier. In specific embodiments, the composition further comprises a chitosan. As discussed above, the drug may be a therapeutic agent or a diagnostic agent. In particular embodiments, the drug is an anticancer agent. Non-limiting examples of anticancer agents include any of the aforementioned agents. In specific embodiments, the anticancer agent is a curcuminoid or emodin.

The present invention also generally concerns methods of delivering a drug and/or nutraceutical to a subject involving administering to the subject an effective amount of a composition that includes a nanoparticle as set forth herein. The subject may be any subject, but in particular embodiments the subject is a mammal. Non-limiting examples of mammals contemplated include mice, rats, rabbits, cats, dogs, sheep, goats, horses, cows, primates, and humans. In specific embodiments, the subject is a human.

The methods of delivering a drug set forth herein may involve delivery of a diagnostic agent or a therapeutic agent. The therapeutic agent, for example, may be delivered for the purpose of treating or preventing a disease or health-related condition in a subject. In particular embodiments, the subject is a human with a disease, and the drug is delivered for the purpose of treating a disease in the subject. The disease may be any disease. Non-limiting examples of types of diseases include cancer, premalignancies, infectious diseases, inflammatory diseases, and degenerative diseases. Non-limiting examples of types of cancer include breast cancer, lung cancer, prostate cancer, ovarian cancer, brain cancer cell, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, intestinal cancer, lymphoma, and leukemia. In specific embodiments, the cancer is breast cancer.

Regarding therapeutic agents, any therapeutic agent is contemplated for inclusion in the nanoparticles of the present invention. In certain embodiments, the therapeutic agent is an anticancer agent. Non-limiting examples of anticancer agents are set forth elsewhere in this specification. In a particular embodiments, the drug is a curcuminoid. In a further particular embodiment, the drug is emodin.

The present invention also concerns methods of preparing a nanoparticle of the present invention that includes the steps of: (a) preparing a composition that includes a SF polypeptide, one or more drugs and/or nutraceuticals, and a carrier; (b) dispensing droplets of the composition of (a) on a surface; (c) lyophilizing the surface of (b) to remove the carrier, wherein dried dots of a composition that include a SF polypeptide and drug and/or nutraceutical are formed on the surface; (d) removing the composition of (c) from the surface, wherein the composition of (c) includes nanoparticles having a diameter of about 1 nm to about 500 nm. In some embodiments, the method includes freezing the surface following dispensing of the droplets of the composition of (a) on the surface, and prior to step (c). The nanoparticles that are obtained in step (d) may optionally be washed in a buffered solution. The dots may be of any size, but in particular embodiments are 500 micrometer to 3 mn in diameter. The nanoparticles that are obtained may be frozen for later use. The may be stored in a dried form at room temperature or in a pharmaceutical carrier. The surface may be composed of any material but in particular embodiments the surface is a glass surface. The drug may be any of the aforementioned agents. In specific embodiments, the drug is an anticancer agent, such as a curcuminoid or emodin. In certain embodiments, the composition of step (a) further includes a chitosan, and the nanoparticles that are formed include a chitosan, a silk fibroin polypeptide, and one or more drugs. In some embodiments the method of preparing the nanoparticle further comprises sonicating the composition of (a). Sonication can be performed for any duration. For example, it can be performed for not greater than 72 hours.

Also included in the present invention are kits that include at least a first sealed container that includes a fibroin polypeptide and a drug and/or nutraceutical. The kit may include any of the aforementioned nanoparticles of the present invention. In particular embodiments, the kit incudes a nanoparticle in the first sealed container that includes an anticancer agent, such as any of the anticancer agents set forth above. In certain embodiments, the anticancer agent is curcumin or emodin.

The kit may optionally include one or more additional items, such as instructions, a syringe, a catheter, a needle, a second sealed container that includes an aqueous solvent such as phosphate buffered saline or normal saline.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Any embodiment of any of the present methods, compositions, nanoparticles, and kits may consist of or consist essentially of—rather than comprise/include/contain/have—the described features and/or steps. Thus, in any of the claims, the term “consisting of” or “consisting essentially of” may be substituted for any of the open-ended linking verbs recited above, in order to change the scope of a given claim from what it would otherwise be using the open-ended linking verb.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1. Curcumin nanoparticle sizes as measured from TEM images. Between 22 to 50 nanoparticles were measured from TEM images for each formulation. *p<0.001 vs. 0.1% SF, ^{†}p<0.001 vs. 0.1% 25:75 SFCS, ^{φ}p<0.05 vs. 10% 25:75 SFCS, ^{‡}p<0.001 vs. 0.1% 50:50 SFCS, ^{#}p<0.001 vs. 10% 75:25 SFCS.

FIG. 2. Curcumin entrapment within nanoparticles (n=3). *p<0.01 vs. 0.1% SF, ^{†}p<0.001 vs. 0.1% SF, ^{‡}p<0.05 vs. 10% SF, ^{φ}p<0.01 vs. 10% SF.

FIGS. 3A, 3B. Cumulative curcumin release from nanoparticles over the period of 8 days (n=3). All blends of SFCS and SF alone were made of (4A) 0.1% solution and (4B) 10% solution.

FIGS. 4A, 4B. Intracellular uptake of curcumin by breast cancer cells as measured by absorbance assay after exposure to curcumin nanoparticles for 4 days (n=3). (5A) MCF-7, *p<0.01 vs. 0.1% SF, ⁵⁵⁴ p<0.001 vs. 10% SF (5B) MDA-MB-453, ^{‡}p<0.001 vs. 0.1% SF, ^{φ}p<0.001 vs. 10% SF, ^{#}p<0.01 vs. 10% SF.

FIGS. 5A, 5B. Intracellular uptake of curcumin by breast cancer cells as measured by fluorescence assay after exposure to curcumin nanoparticles for 4 days (n=3). (6A) MCF-7, *p<0.01 vs. 0.1% SF, ^{†}p<0.05 vs. 10% SF (6B) MDA-MB-453, ^{‡}p<0.001 vs. 0.1% SF, ^{φ}p<0.001 vs. 10% SF.

FIGS. 6A, 6B. Cell viability measured by MTT assay after exposure to curcumin nanoparticles for 4 days (n=3). (7A) MCF-7, *p<0.001 vs. control, ^{†}p<0.01 vs. 0.1% 25:75 SFCS, ^{‡}p<0.05 vs. 10% 25:75 SFCS and 10% 50:50 SFCS (7B) MDA-MB-453, *p<0.01 vs. control, ^{†}p<0.01 vs. 0.1% 50:50 SFCS, ^{‡}p<0.05 vs. 10% 25:75 SFCS and 10% 50:50 SFCS.

FIG. 7. Nanoparticle Size of nanocurcumin particles. *indicates p<0.001 as compared to 75:25 SFCS and 0.1% SF with curcumin; @ indicates p<0.001 as compared to 75:25 SFCS and 0.1% SF with curcumin; # indicates p<0.001 as compared to 0.1% SF with curcumin; $ indicates p<0.0001 as compared to 25:75 and 75:25 SFCS without curcumin; & indicates p<0.001 as compared to the same time point with curcumin.

FIG. 8. Enhanced curcumin entrapment with 0.1% SF. *P<0.05 vs. 0.1% SF.

FIG. 9. Curcumin release from nanoparticles. Burst release observed with SFCS blends; slower but higher release for 0.1% SF.

FIG. 10. Intracellular uptake of nanocurcumin into breast cancer cells—absorbance. Only 0.1% SF showed significant uptake of curcumin; uptake was similar for MCF-7 and MDA-MB-453 cells. *P<0.001, 0.1% SF vs. all other blends.

FIG. 11. Intracellular uptake of nanocurcumin into breast cancer cells—fluorescence. Only 0.1% SF showed significant update of curcumin; uptake was similar for MCF-7 and MDA-MB-453 cells. *P<0.001, 0.1% SF vs. all other blends.

FIG. 12. Standards for cell viability assay of breast cancer cells incubated with nanocurcumin. Seeded cells in 96-well plate; incubated for 4 days; performed MTT assay for cell viability.

FIG. 13. Efficacy of nanocurcumin on cells. MTT assay used for cell viability; 0.1% SF showed significant decrease in cell number compared to other blends; efficacy was similar for MCF-7 and MDA-MB-453 cells. †P<0.01 vs. 0.1% SF, tP<0.05 vs. 0.1% SF.

FIG. 14. Reduced liposome size by sonication.

FIG. 15. Higher emodin entrapment and release with sonication.

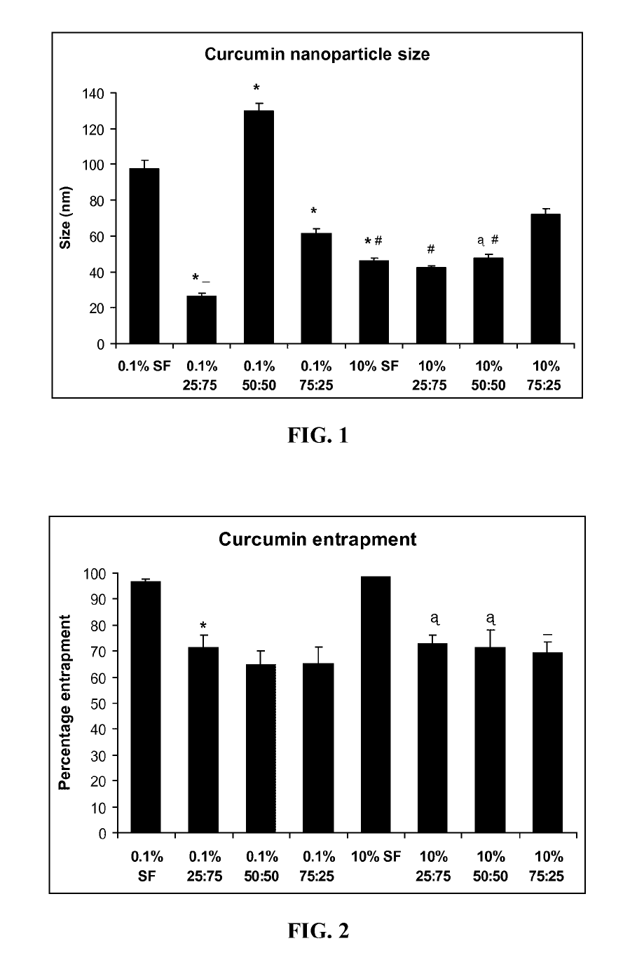

### DESCRIPTION OF ILLUSTRATIVE EMBODIMENTS

The present invention is based on the finding that silk fibroin nanoparticles can be applied in the delivery of therapeutic agents, diagnostic agents, and/or nutraceuticals to a subject. The nanoparticles result in improved bioavailability, improved efficacy, and reduced risk of toxicity. For example, curcumin nanoparticles can be delivered to the site of a tumor in a manner that allows for long-term drug delivery and retention of the drug in the diseased cells. Silk fibroin provides a delivery mechanism for any therapeutic or diagnostic agent to a cell of interest. It is a carrier system that delivers the pharmaceutical or nutraceutical to the respective cells and retains it at the diseased site so that bioavailability at the site of disease is increased with resulting improved efficacy and reduced systemic toxicity.

### A. Silk Fibroin Polypeptides

1. Silk Fibroin Generally

Silk, as the term is generally known in the art, means a filamentous fiber product secreted by an organism. Non-limiting examples of such organisms include a silkworm or a spider. Silks produced from insects, namely (i) Bombyx mori silkworms, and (ii) the glands of spiders, typically Nephilia clavipes, are the most often studied forms of the material; however, hundreds to thousands of natural variants of silk exist in nature. Fibroin is produced and secreted by a silkworm's two silk glands.

Silkworm silk has been used in biomedical applications for over 1,000 years. The Bombyx mori species of silkworm produces a silk fiber (known as a “bave”) and uses the fiber to build its cocoon. The bave, as produced, includes two fibroin filaments or “broins,” which are surrounded with a coating of gum, known as sericin—the silk fibroin filament possesses significant mechanical integrity. When silk fibers are harvested for producing yarns or textiles, the sericin is partially dissolved and then resolidified to create a larger silk fiber structure having more than two broins mutually embedded in a sericin coating.

As used herein, the term “silk fibroin” pertains to silkworm fibroin. SF may be obtained from any source known to those of ordinary skill in the art. For example, SF may be obtained from a solution containing a dissolved silkworm silk from Bombyx mori. In the alternative, the SF suitable for use in the present invention can be obtained from a solution containing a genetically engineered silk.

The SF can be prepared by any conventional method known to one skilled in the art. For example, B. mori cocoons may be boiled in an aqueous solution. The cocoons are rinsed, for example, with water to extract the sericin proteins and the extracted silk is dissolved in an aqueous salt solution. The salt is consequently removed using, for example, dialysis. The SF may be produced using organic solvents. Such methods have been described, for example, in Li et al. (2001); Nazarov et al. (2004). SF may also be obtained from any of a number of commercial sources known to those of ordinary skill in the art.

Additional information concerning the production of silk fibroin can be found in U.S. Patent App. Pub. No. 20080176960, 20070187862, 20060019348, 20050260706, 20030165548, herein specifically incorporated by reference.

2. Polypeptides

In certain embodiments, the present invention concerns nanoparticles comprising at least one SF polypeptide. As used herein, the term “polypeptide” refers to a consecutive series of two or more amino acids.

In certain embodiments the size of at least SF polypeptide may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 260, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues, or any range of amino acid residues derivable therein (e.g., about 200 to about 2500 amino acid residues). For example, the SF polypeptide may include between 10 and 500 amino acid residues, between 20 and 200 amino acid residues, or between 30 and 150 amino acid residues.

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moiety. In particular embodiments, the sequence of residues of the protein or peptide may be interrupted by one or more non-amino acid moieties.

Accordingly, the term “polypeptide” encompasses amino acid sequences comprising at least one of the 20 common amino acids found in naturally occurring proteins, or at least one modified or unusual amino acid, including but not limited to Aad, 2-Aminoadipic acid; EtAsn, N-Ethylasparagine; Baad, 3-Aminoadipic acid, Hyl, Hydroxylysine; Bala, β-alanine, β-Amino-propionic acid; AHyl, allo-Hydroxylysine; Abu, 2-Aminobutyric acid; 3Hyp, 3-Hydroxyproline; 4Abu, 4-Aminobutyric acid, piperidinic acid; 4Hyp, 4-Hydroxyproline; Acp, 6-Aminocaproic acid, Ide, Isodesmosine; Ahe, 2-Aminoheptanoic acid; AIle, allo-Isoleucine; Aib, 2-Aminoisobutyric acid; MeGly, N-Methylglycine, sarcosine; Baib, 3-Aminoisobutyric acid; MeIle, N-Methylisoleucine; Apm, 2-Aminopimelic acid; MeLys, 6-N-Methyllysine; Dbu, 2,4-Diaminobutyric acid; MeVal, N-Methylvaline; Des, Desmosine; Nva, Norvaline; Dpm, 2,2′-Diaminopimelic acid; Nle, Norleucine; Dpr, 2,3-Diaminopropionic acid; Orn, Ornithine; and EtGly, N-Ethylglycine.

Proteins or peptides may be made by any technique known to those of skill in the art, including the expression of polypeptides through standard molecular biological techniques, the isolation of polypeptides from natural sources, or the chemical synthesis of polypeptides. Alternatively, various commercial preparations of SF polypeptides are known to those of skill in the art.

### B. Drugs

1. In General

The nanoparticles of the present invention include one or more drugs. A “drug” as used herein refers to a diagnostic or therapeutic agent. A “therapeutic agent” as used herein refers to any agent that can be administered to a subject for the purpose of obtaining a therapeutic benefit of a disease or health-related condition. For example, nanoparticles that include a therapeutic agent may be administered to a subject for the purpose of reducing the size of a tumor, reducing or inhibiting local invasiveness of a tumor, or reducing the risk of development of metastases.

A “diagnostic agent” as used herein refers to any agent that can be administered to a subject for the purpose of diagnosing a disease or health-related condition in a subject. Diagnosis may involve determining whether a disease is present, whether a disease has progressed, or any change in disease state.

The drug may be a small molecule, a peptide, a protein, a polypeptide, an antibody, an antibody fragment, a DNA, or an RNA. The therapeutic agent may be a siRNA, miRNA, or shRNA.

The therapeutic agent or diagnostic agent can be any such agent known to those of ordinary skill in the art. For example, the therapeutic agent may be an anti-inflammatory agent, an anti-infective agent, an agent that can be applied in the treatment of a hyperproliferative disease such as cancer, an agent that can be applied in the treatment of a degenerative disease, and so forth.

Other examples of therapeutic agents include, but are not limited to, agents for the prevention of restenosis, agents for treating renal disease, agents used for intermittent claudication, agents used in the treatment of hypotension and shock, angiotensin converting enzyme inhibitors, antianginal agents, anti-arrhythmics, anti-hypertensive agents, antiotensin ii receptor antagonists, antiplatelet drugs, b-blockers b1 selective, beta blocking agents, botanical product for cardiovascular indication, calcium channel blockers, cardiovascular/diagnostics, central alpha-2 agonists, coronary vasodilators, diuretics and renal tubule inhibitors, neutral endopeptidase/angiotensin converting enzyme inhibitors, peripheral vasodilators, potassium channel openers, potassium salts, anticonvulsants, antiemetics, antinauseants, anti-parkinson agents, antispasticity agents, cerebral stimulants, agents that can be applied in the treatment of trauma, agents that can be applied in the treatment of Alzheimer disease or dementia, agents that can be applied in the treatment of migraine, agents that can be applied in the treatment of neurodegenerative diseases, agents that can be applied in the treatment of kaposi's sarcoma, agents that can be applied in the treatment of AIDS, cancer chemotherapeutic agents, agents that can be applied in the treatment of immune disorders, agents that can be applied in the treatment of psychiatric disorders, analgesics, epidural and intrathecal anesthetic agents, general, local, regional neuromuscular blocking agents sedatives, preanesthetic adrenal/acth, anabolic steroids, agents that can be applied in the treatment of diabetes, dopamine agonists, growth hormone and analogs, hyperglycemic agents, hypoglycemic agents, oral insulins, largevolume parenterals (lvps), lipid-altering agents, metabolic studies and inborn errors of metabolism, nutrients/amino acids, nutritional lvps, obesity drugs (anorectics), somatostatin, thyroid agents, vasopressin, vitamins, corticosteroids, mucolytic agents, pulmonary anti-inflammatory agents, pulmonary surfactants, antacids, anticholinergics, antidiarrheals, antiemetics, cholelitholytic agents, inflammatory bowel disease agents, irritable bowel syndrome agents, liver agents, metal chelators, miscellaneous gastric secretory agents, pancreatitis agents, pancreatic enzymes, prostaglandins, prostaglandins, proton pump inhibitors, sclerosing agents, sucralfate, anti-progestins, contraceptives, oral contraceptives, not oral dopamine agonists, estrogens, gonadotropins, GNRH agonists, GHRH antagonists, oxytocics, progestins, uterine-acting agents, anti-anemia drugs, anticoagulants, antifibrinolytics, antiplatelet agents, antithrombin drugs, coagulants, fibrinolytics, hematology, heparin inhibitors, metal chelators, prostaglandins, vitamin K, anti-androgens, aminoglycosides, antibacterial agents, sulfonamides, cephalosporins, clindamycins, dermatologics, detergents, erythromycins, anthelmintic agents, antifungal agents, antimalarials, antimycobacterial agents, antiparasitic agents, antiprotozoal agents, antitrichomonads, antituberculosis agents, immunomodulators, immunostimulatory agents, macrolides, antiparasitic agents, corticosteroids, cyclooxygenase inhibitors, enzyme blockers, immunomodulators for rheumatic diseases, metalloproteinase inhibitors, nonsteroidal anti-inflammatory agents, analgesics, antipyretics, alpha adrenergic agonists/blockers, antibiotics, antivirals, beta adrenergic blockers, carbonic anhydrase inhibitors, corticosteroids, immune system regulators, mast cell inhibitors, nonsteroidal anti-inflammatory agents, prostaglandins, and proteolytic enzymes.

Examples of diagnostic agents include, but are not limited to, magnetic resonance image enhancement agents, positron emission tomography products, radioactive diagnostic agents, radioactive therapeutic agents, radio-opaque contrast agents, radiopharmaceuticals, ultrasound imaging agents, angiographic diagnostic agents, and other reporter agents.

In particular embodiments, the therapeutic agent is a chemotherapeutic agent. A wide variety of chemotherapeutic agents may be used in accordance with the present invention. The term “chemotherapy” refers to the use of drugs to treat cancer. A “chemotherapeutic agent” (or “anticancer agent”) is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1; dynemicin, including dynemicin A); bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; a curcuminoid, emodin, and pharmaceutically acceptable salts, acids or derivatives of any of the above.

2. Curcuminoids

In particular embodiments, the therapeutic agent is a curcuminoid. As used herein, curcuminoids are those compounds which due to their structural similarity to curcumin, exhibit anti-proliferative or pro-apoptotic effects on cancer cells similar to that of curcumin. As used herein curcumin is also known as diferuloylmethane or (E,E)-1,7-bis(4-hydroxy-3-methoxyphenyl)-1,6-heptadiene-3,5-dione. Curcuminoids, or analogs of curcumin, which may have anti-cancer effects similar to curcumin include, for example, Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis(4-hydroxy-3-methoxyphenyl)-1,6-heptadiene-3,5-dione (curcumin 1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin), 1,1-bis(phenyl)-1,3,8,10 undecatetraene-5,7-dione (cinnamyl curcumin) and the like (Araujo and Leon, 2001; Lin et al., 2001; John et al., 2002; see also Ishida et al., 2002). Curcumin analogs may also include isomers of curcumin, such as the (Z,E) and (Z,Z) isomers of curcumin. In a related embodiment, curcumin metabolites which have anti-cancer effects similar to curcumin can also be used in the present invention. Known curcumin metabolites include glucoronides of tetrahydrocurcumin and hexahydrocurcumin, and dihydroferulic acid. In certain embodiments, curcumin analogs or metabolites can be formulated as metal chelates, especially copper chelates. Other appropriate derivatives of curcumin, curcumin analogues and curcumin metabolites appropriate for use in the present invention will be apparent to one of skill in the art.

Curcuminoids may be derived from a natural source, such as the perennial herb Curcuma longa L., which is a member of the Zingiberaceae family. The spice turmeric is extracted from the rhizomes of Curcuma longa L. and has long been associated with traditional-medicine treatments used in Hindu and Chinese medicine. Turmeric was administered orally or topically in these traditional treatment methods. Curcuminoids may also be chemically synthesized or obtained from commercial sources.

### C. Chitosan and Analogs Thereof

The nanoparticles of the present invention include chitosan as a component. Generally, chitosans are a family of cationic, binary hetero-polysaccharides composed of (1→4)-linked 2-acetamido-2-deoxy-β-D-glucose (GlcNAc, A-unit) and 2-amino-2-deoxy-β-D-glucose, (GlcN; D-unit) (Varum et al., 1991). The chitosan has a positive charge, stemming from the de-acetylated amino group (—NH₃⁺). Chitosan, chitosan derivatives or salts (e.g., nitrate, phosphate, sulphate, hydrochloride, glutamate, lactate or acetate salts) of chitosan may be used and are included within the meaning of the term “chitosan.” As used herein, the term “chitosan derivatives” is intended to include ester, ether, or other derivatives formed by bonding of acyl and/or alkyl groups with —OH groups, but not the NH₂ groups, of chitosan. Examples are O-alkyl ethers of chitosan and O-acyl esters of chitosan. Modified chitosans, particularly those conjugated to polyethylene glycol, are also considered “chitosan derivatives.” Many chitosans and their salts and derivatives are commercially available (e.g., SigmaAldrich, Milwaukee, Wis.).

Methods of preparing chitosans and their derivatives and salts are also know, such as boiling chitin in concentrated alkali (50% w/v) for several hours—this produces chitosan wherein 70-75% of the N-acetyl groups have been removed. A non-limiting example of a chitosan, wherein all of the N-acetyl groups have been removed, is shown in formula (III) below.

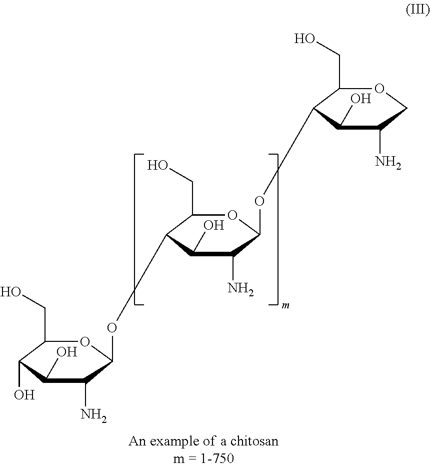

Chitosans may be obtained from any source known to those of ordinary skill in the art. For example, chitosans may be obtained from commercial sources. Chitosans may be obtained from chitin, the second most abundant biopolymer in nature. Chitosan is prepared by N-deacetylation of chitin. Chitosan is commercially available in a wide variety of molecular weight (e.g., 10-1000 kDa) and usually has a degree of deacetylation ranging between 70%-90%.

The chitosan (or chitosan derivative or salt) used preferably has a molecular weight of 4,000 Dalton or more, preferably in the range 25,000 to 2,000,000 Dalton, and most preferably about 50,000 to 300,000 Dalton. Chitosans of different molecular weights can be prepared by enzymatic degradation of high molecular weight chitosan using chitosanase or by the addition of nitrous acid. Both procedures are well known to those skilled in the art and are described in various publications (Allan and Peyron, 1995; Domard and Cartier, 1989). The chitosan is water-soluble and may be produced from chitin by deacetylation to a degree of greater than 40%, preferably between 50% and 98%, and more preferably between 70% and 90%.

Some methods of producing chitosan involve recovery from microbial biomass, such as the methods taught by U.S. Pat. No. 4,806,474 and U.S. Patent Application No. 20050042735, herein incorporated by reference. Another method, taught by U.S. Pat. No. 4,282,351, teaches only how to create a chitosan-beta-glucan complex.

Chitosan derivatives are also suitable for use in this invention. Suitable chitosan derivatives include, without limitation, esters, ethers or other derivatives formed by bonding acyl and/or alkyl groups with the hydroxyl groups, but not the amino groups of chitosan. Examples include O-alkyl ethers of chitosan and O-acyl esters of chitosan.

The chitosan, chitosan derivative or salt used in the present invention may be water soluble. Chitosan glutamate is water soluble. By “water soluble” it is meant that the chitosan, chitosan derivative or salt dissolves in water at an amount of at least 10 mg/ml at room temperature and atmospheric pressure.

Additional information regarding chitosan and chitosan derivatives can be found in U.S. Patent App. Pub. Nos. 20070167400, 20070116767, 20070311468, 20060277632, 20060189573, 20060094666, 20050245482, 20050226938, 20040247632, and 20030129730, each of which is herein specifically incorporated by reference.

### D. Methods of Making Nanoparticles Comprising a Silk Fibroin Polypeptide and a Drug

1. Preparation of Silk Fibroin Nanoparticles that Include a Drug

Silk fibroin polypeptide-containing nanoparticles may be formulated by mixing one or more therapeutic agents with a SF-containing solution. Alternatively, a drug containing particle can be coated onto the pre-formed drug-containing particle. Any pharmaceutical carrier can be used that does not dissolve the silk fibroin. Examples of methods for formation of silk fibroin nanoparticles are discussed in the Example section below.

In brief, raw silk can be obtained and degummed using any method known to those of ordinary skill in the art. For example, the raw silk may be degummed in 0.25% sodium carbonate and 0.25% solidium dodecylsulfate, and then dissolved in calcium nitrate tetrahydrate and methanol solution. Drug can be suspended in a composition that includes the silk fibroin polypeptide. The nanoparticles that form in the composition can be extracted using any method known to those of ordinary skill in the art. For example, the drug suspension can be dispersed on a glass slide, the slide can be frozen, and then lyophilized. Dry dots containing SFCS coated drug nanoparticles can then be scrated off the slides and collected in an appropriate container. The nanoparticles can then be washed, and stored for later use.

2. Preparation of Silk Fibroin—Chitosan Nanoparticles that Include a Drug

In some embodiments of the present invention, the nanoparticles are formed of a combination of a silk fibroin polypeptide and a chitosan. Information regarding SF has been previously discussed. For example, raw silk can be obtained and degummed using any method known to those of ordinary skill in the art.

The preferred process for preparing the nanoparticles of the invention is by mixing together the ingredients. Information regarding preparation of compositions that include SF and a chitosan can be found in Gobin et al. (2005). For example, the raw silk may be degummed in 0.25% sodium carbonate and 0.25% solidium dodecylsulfate, and then dissolved in calcium nitrate tetrahydrate and methanol solution. Chitosan can be dissolved in a suitable solvent, such as 2% acetic acid, and then blended with a mixture of SF in an appropriate solvent, such as calcium nitrate tetrahydrate and methanol solution.

Examples are set forth in detail in the specification below. In this process, chitosan (such as a powder of chitosan or a derivative thereof or a salt of chitosan or a salt of a derivative of chitosan) is dissolved in a suitable solvent to form a solution. For example, the solvent may be water, acetic acid, or hydrochloric acid. The chitosan-containing solution that is formed may optionally be centrifuged to remove contaminants, although removal of all contaminants is not required. The pH of the chitosan solution may then be adjusted such that the pH is in a range of about 3.5 to about 5.5.

A blend of silk fibroin polypeptide and chitosan may then be created. One or more drugs can optionally be added to the chitosan-silk fibroin blend.

The silk fibroin—chitosan (SFCT) blend can then be dialyzed against water and filtered. The blend can then be combined with a composition that includes one or more drugs to form nanoparticles, and the nanoparticles can be isolated from the composition using any method known to those of ordinary skill in the art.

3. Additives

Additives suitable for use with the present invention include biologically or pharmaceutically active compounds. Examples of biologically active compounds include, but are not limited to: cell attachment mediators, such as collagen, elastin, fibronectin, vitronectin, laminin, proteoglycans, or peptides; biologically active ligands; and substances that enhance or exclude particular varieties of cellular or tissue ingrowth.

Biocompatible polymers can be added to the silk fibroin containing nanoparticles of the present invention. Biocompatible polymers useful in the present invention include, for example, polyethylene oxide (PEO) (U.S. Pat. No. 6,302,848), polyethylene glycol (PEG) (U.S. Pat. No. 6,395,734), collagen (U.S. Pat. No. 6,127,143), fibronectin (U.S. Pat. No. 5,263,992), keratin (U.S. Pat. No. 6,379,690), polyaspartic acid (U.S. Pat. No. 5,015,476), polylysine (U.S. Pat. No. 4,806,355), alginate (U.S. Pat. No. 6,372,244), chitosan (U.S. Pat. No. 6,310,188), chitin (U.S. Pat. No. 5,093,489), hyaluronic acid (U.S. Pat. No. 6,387,413), pectin (U.S. Pat. No. 6,325,810), polycaprolactone (U.S. Pat. No. 6,337,198), polylactic acid (U.S. Pat. No. 6,267,776), polyglycolic acid (U.S. Pat. No. 5,576,881), polyhydroxyalkanoates (U.S. Pat. No. 6,245,537), dextrans (U.S. Pat. No. 5,902,800), and polyanhydrides (U.S. Pat. No. 5,270,419).

4. Purification

The nanoparticles can be purified using any method known to those of ordinary skill in the art. In particular embodiments, the nanoparticles may be purified by centrifugation and removal of supernatant. For example, centrifugation may be at 12000 rpm for about 30 min to about 60 min. Centrifugation may be repeated once, or more than once. In particular embodiments, centrifugation is repeated three times.

5. Analysis of Formed Nanoparticles

Nanoparticles that are formed by the present methods can be analyzed using any method and technique known to those of ordinary skill in the art. For example, particle size may be measured by dynamic light scattering or by transmission electron microscopy.

In some embodiments, nanoparticle size is heterogeneous and poorly defined. If desired, nanoparticle size may be reduced using any method known to those of ordinary skill in the art. The nanoparticle size can be controlled using standard techniques such as sieving.

6. Storage

The nanoparticles may be stored using any method known to those of ordinary skill in the art. The nanoparticles may be stored at 4° C. until ready for use. In some embodiments, the nanoparticles are stored at room temperature. Nanoparticles of the present invention may be stored in a dry form and reconstituted for later use, or they can be stored dispersed in a solution.

7. Optional Ingredients

The particles of the present invention may optionally include one or more additional ingredients. Examples of additional ingredients include, but are not limited to, sugars such as sucrose and trehalose; polyols such as mannitol and sorbitol; and surfactants such as polysorbates; amino acids such as glycine; and polyethylene glycol. The total amount of additional ingredients may be up to a total of about 10% by weight of the nanoparticle.

8. Targeting Moieties

In certain embodiments of the present invention, the nanoparticle further includes a targeting moiety. A “targeting moiety” is a term that encompasses various types of affinity reagents that may be used to enhance the localization or binding of a substance to a particular location in an animal, including organs, tissues, particular cell types, diseased tissues or tumors. A targeting moiety is still considered to selectively bind even if it also binds to other proteins that are not substantially homologous with the target so long as such proteins share homology with a fragment or domain of the peptide target of the antibody. In this case, it would be understood that target moiety binding to the target is still selective despite some degree of cross-reactivity. Typically, the degree of cross-reactivity can be determined and differentiated from binding to the target.

Targeting moieties may include peptides, peptide mimetics, polypeptides, antibodies, antibody-like molecules, nucleic acids, aptamers, and fragments thereof. Targeting moieties also include small molecules. The targeting moiety may be covalently bound to the nanoparticle or noncovalently bound to the nanoparticle.

Other materials or substances which may serve as targeting ligands include, for example, proteins, including antibodies, antibody fragments, hormones, hormone analogues, glycoproteins and lectins, peptides, polypeptides, amino acids, sugars, saccharides, including monosaccharides and polysaccharides, carbohydrates, vitamins, steroids, steroid analogs, hormones, cofactors, and genetic material, including nucleosides, nucleotides, nucleotide acid constructs, petptide nucleic acids (PNA), aptamers, and polynucleotides. Other targeting ligands in the present invention include cell adhesion molecules (CAM), among which are, for example, cytokines, integrins, cadherins, immunoglobulins and selectin. The pharmaceutical formulations of the present invention may also encompass precursor targeting ligands. A precursor to a targeting ligand refers to any material or substance which may be converted to a targeting ligand. Such conversion may involve, for example, anchoring a precursor to a targeting ligand. Exemplary targeting precursor moieties include maleimide groups, disulfide groups, such as ortho-pyridyl disulfide, vinylsulfone groups, azide groups, and [agr]-iodo acetyl groups.

### E. Lipid Compositions and Liposomes

In some embodiments set forth herein, the nanoparticle includes a lipid. The nanoparticle may be a liposome that is coated with a silk fibroin polypeptide or a composition that includes a silk fibroin polypeptide.

Lipid complexes and liposomes can be formed using any method known to those of ordinary skill in the art. Selection of the appropriate lipids for such composition is governed by the factors of: (1) liposome stability, (2) phase transition temperature, (3) charge, (4) non-toxicity to mammalian systems, (5) encapsulation efficiency, (6) lipid mixture characteristics. It is expected that one of skill in the art who has the benefit of this disclosure could formulate liposomes according to the present invention which would optimize these factors. The vesicle-forming lipids of this type are preferably ones having two hydrocarbon chains, typically acyl chains, and a head group, either polar or nonpolar. The hydrocarbon chains may be saturated or have varying degrees of unsaturation. There are a variety of synthetic vesicle-forming lipids and naturally-occurring vesicle-forming lipids, including the sphingolipids, ether lipids, sterols, phospholipids, particularly the phosphoglycerides, and the glycolipids, such as the cerebrosides and gangliosides.

Phosphoglycerides include phospholipids such as phosphatidylcholine, phosphatidylethanolamine, phosphatidic acid, phosphatidylinositol, phosphatidylserine phosphatidylglycerol and diphosphatidylglycerol (cardiolipin), where the two hydrocarbon chains are typically between about 14-22 carbon atoms in length, and have varying degrees of unsaturation. As used herein, the abbreviation “PC” stands for phosphatidylcholine, and “PS” stand for phosphatidylserine. Lipids containing either saturated and unsaturated fatty acids are widely available to those of skill in the art. Additionally, the two hydrocarbon chains of the lipid may be symmetrical or asymmetrical. The above-described lipids and phospholipids whose acyl chains have varying lengths and degrees of saturation can be obtained commercially or prepared according to published methods.

Exemplary phosphatidylcholines include dilauroyl phophatidylcholine, dimyristoylphophatidylcholine, dipalmitoylphophatidylcholine, distearoylphophatidyl-choline, diarachidoylphophatidylcholine, dioleoylphophatidylcholine, dilinoleoyl-phophatidylcholine, dierucoylphophatidylcholine, palmitoyl-oleoyl-phophatidylcholine, egg phosphatidylcholine, myristoyl-palmitoylphosphatidylcholine, palmitoyl-myristoyl-phosphatidylcholine, myristoyl-stearoylphosphatidylcholine, palmitoyl-stearoyl-phosphatidylcholine, stearoyl-palmitoylphosphatidylcholine, stearoyl-oleoyl-phosphatidylcholine, stearoyl-linoleoylphosphatidylcholine and palmitoyl-linoleoyl-phosphatidylcholine. Assymetric phosphatidylcholines are referred to as 1-acyl, 2-acyl-sn-glycero-3-phosphocholines, wherein the acyl groups are different from each other. Symmetric phosphatidylcholines are referred to as 1,2-diacyl-sn-glycero-3-phosphocholines. As used herein, the abbreviation “PC” refers to phosphatidylcholine. The phosphatidylcholine 1,2-dimyristoyl-sn-glycero-3-phosphocholine is abbreviated herein as “DMPC.” The phosphatidylcholine 1,2-dioleoyl-sn-glycero-3-phosphocholine is abbreviated herein as “DOPC.” The phosphatidylcholine 1,2-dipalmitoyl-sn-glycero-3-phosphocholine is abbreviated herein as “DPPC.”

In general, saturated acyl groups found in various lipids include groups having the trivial names propionyl, butanoyl, pentanoyl, caproyl, heptanoyl, capryloyl, nonanoyl, capryl, undecanoyl, lauroyl, tridecanoyl, myristoyl, pentadecanoyl, palmitoyl, phytanoyl, heptadecanoyl, stearoyl, nonadecanoyl, arachidoyl, heneicosanoyl, behenoyl, trucisanoyl and lignoceroyl. The corresponding IUPAC names for saturated acyl groups are trianoic, tetranoic, pentanoic, hexanoic, heptanoic, octanoic, nonanoic, decanoic, undecanoic, dodecanoic, tridecanoic, tetradecanoic, pentadecanoic, hexadecanoic, 3,7,11,15-tetramethylhexadecanoic, heptadecanoic, octadecanoic, nonadecanoic, eicosanoic, heneicosanoic, docosanoic, trocosanoic and tetracosanoic. Unsaturated acyl groups found in both symmetric and asymmetric phosphatidylcholines include myristoleoyl, palmitoleyl, oleoyl, elaidoyl, linoleoyl, linolenoyl, eicosenoyl and arachidonoyl. The corresponding IUPAC names for unsaturated acyl groups are 9-cis-tetradecanoic, 9-cis-hexadecanoic, 9-cis-octadecanoic, 9-trans-octadecanoic, 9-cis-12-cis-octadecadienoic, 9-cis-12-cis-15-cis-octadecatrienoic, 11-cis-eicosenoic and 5-cis-8-cis-11-cis-14-cis-eicosatetraenoic.

Exemplary phosphatidylethanolamines include dimyristoyl-phosphatidylethanolamine, dipalmitoyl-phosphatidylethanolamine, distearoyl-phosphatidylethanolamine, dioleoyl-phosphatidylethanolamine and egg phosphatidylethanolamine. Phosphatidylethanolamines may also be referred to under IUPAC naming systems as 1,2-diacyl-sn-glycero-3-phosphoethanolamines or 1-acyl-2-acyl-sn-glycero-3-phosphoethanolamine, depending on whether they are symmetric or assymetric lipids.

Exemplary phosphatidic acids include dimyristoyl phosphatidic acid, dipalmitoyl phosphatidic acid and dioleoyl phosphatidic acid. Phosphatidic acids may also be referred to under IUPAC naming systems as 1,2-diacyl-sn-glycero-3-phosphate or 1-acyl-2-acyl-sn-glycero-3-phosphate, depending on whether they are symmetric or assymetric lipids.

Exemplary phosphatidylserines include dimyristoyl phosphatidylserine, dipalmitoyl phosphatidylserine, dioleoylphosphatidylserine, distearoyl phosphatidylserine, palmitoyl-oleylphosphatidylserine and brain phosphatidylserine. Phosphatidylserines may also be referred to under IUPAC naming systems as 1,2-diacyl-sn-glycero-3-[phospho-L-serine] or 1-acyl-2-acyl-sn-glycero-3-[phospho-L-serine], depending on whether they are symmetric or assymetric lipids. As used herein, the abbreviation “PS” refers to phosphatidylserine.

Exemplary phosphatidylglycerols include dilauryloylphosphatidylglycerol, dipalmitoylphosphatidylglycerol, distearoylphosphatidylglycerol, dioleoyl-phosphatidylglycerol, dimyristoylphosphatidylglycerol, palmitoyl-oleoyl-phosphatidylglycerol and egg phosphatidylglycerol. Phosphatidylglycerols may also be referred to under IUPAC naming systems as 1,2-diacyl-sn-glycero-3-[phospho-rac-(1-glycerol)] or 1-acyl-2-acyl-sn-glycero-3-[phospho-rac-(1-glycerol)], depending on whether they are symmetric or assymetric lipids. The phosphatidylglycerol 1,2-dimyristoyl-sn-glycero-3-[phospho-rac-(1-glycerol)] is abbreviated herein as “DMPG”. The phosphatidylglycerol 1,2-dipalmitoyl-sn-glycero-3-(phospho-rac-1-glycerol) (sodium salt) is abbreviated herein as “DPPG”.

Suitable sphingomyelins might include brain sphingomyelin, egg sphingomyelin, dipalmitoyl sphingomyelin, and distearoyl sphingomyelin.

Other suitable lipids include glycolipids, sphingolipids, ether lipids, glycolipids such as the cerebrosides and gangliosides, and sterols, such as cholesterol or ergosterol. As used herein, the term cholesterol is sometimes abbreviated as “Chol.”

Additional lipids suitable for use in liposomes are known to persons of skill in the art and are cited in a variety of sources, such as 1998 McCutcheon's Detergents and Emulsifiers, 1998 McCutcheon's Functional Materials, both published by McCutcheon Publishing Co., New Jersey, and the Avanti Polar Lipids, Inc. Catalog.

Suitable lipids for use in the present invention will have sufficient long-term stability to achieve an adequate shelf-life. Factors affecting lipid stability are well-known to those of skill in the art and include factors such as the source (e.g. synthetic or tissue-derived), degree of saturation and method of storage of the lipid.

### F. Treatment and Prevention of Disease

1. Definitions

“Treatment” and “treating” as used herein refer to administration or application of a therapeutic agent to a subject or performance of a procedure or modality on a subject for the purpose of obtaining a therapeutic benefit of a disease or health-related condition. For example, nanoparticles that include a therapeutic agent may be administered to a subject for the purpose of reducing the size of a tumor, reducing or inhibiting local invasiveness of a tumor, or reducing the risk of development of metastases.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, reduction in the size of a tumor.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition. For example, a subject at risk of developing cancer may be administered an effective amount of a composition comprising nanoparticles of the present invention to reduce the risk of development of the cancer compared to the risk in a subject that did not receive nanoparticles.

“Determining prognosis” as used herein refers to predicting the likelihood that a subject with have a certain course or outcome of a disease. For example, in some embodiments determining prognosis involves determining likelihood of reduced survival or likelihood of tumor growth.

2. Diseases to be Treated or Prevented

Certain embodiments of the present invention concern methods of treating or preventing disease in a subject involving administration of nanoparticles of the present invention. The disease may be any disease that can affect a subject. Non-limiting examples of diseases include hyperproliferative disease, an inflammatory disease, a degenerative disease, or an infectious disease. In particular embodiments, the disease is a hyperproliferative disease. In more particular embodiments, the disease is cancer.

The cancer can be any cancer. For example, the cancer may be a solid tumor, metastatic cancer, or non-metastatic cancer. In certain embodiments, the cancer may originate in the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In certain embodiments, the cancer is human ovarian cancer. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; muco epidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, and/or a neurodegenerative disease).

### G. Pharmaceutical Preparations

Certain of the methods set forth herein pertain to methods involving the administration of a pharmaceutically effective amount of a composition comprising nanoparticles of the present invention.

1. Compositions

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Except insofar as any conventional carrier is incompatible with the active ingredient, its use in the therapeutic or pharmaceutical compositions is contemplated. The compositions used in the present invention may comprise different types of carriers depending on whether it is to be administered in solid, liquid or aerosol form, and whether it need to be sterile for such routes of administration as injection.

The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions, and these are discussed in greater detail below. For human administration, preparations should meet sterility, pyrogenicity, general safety and purity standards as required by FDA Office of Biologics standards.

The compositions comprising nanoparticles may be extensively dialyzed to remove undesired small molecular weight molecules and/or lyophilized for more ready formulation into a desired vehicle, where appropriate. The active compounds will then generally be formulated for administration by any known route, such as parenteral administration. Methods of administration are discussed in greater detail below.

The present invention contemplates methods using compositions that are sterile solutions for intravascular injection or for application by any other route as discussed in greater detail below. A person of ordinary skill in the art would be familiar with techniques for generating sterile solutions for injection or application by any other route. Sterile injectable solutions are prepared by incorporating the active compounds in the required amount in the appropriate solvent with various of the other ingredients familiar to a person of skill in the art.

The formulation of the composition may vary depending upon the route of administration. For parenteral administration in an aqueous solution, for example, the solution should be suitably buffered if necessary and the liquid diluent first rendered isotonic with sufficient saline or glucose. In this connection, sterile aqueous media which can be employed will be known to those of skill in the art in light of the present disclosure.

In addition to the compounds formulated for parenteral administration, such as intravenous or intramuscular injection, other pharmaceutically acceptable forms include, formulations for administration via an implantable drug delivery device, and any other form. One may also use nasal solutions or sprays, aerosols or inhalants in the present invention.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. A person of ordinary skill in the art would be familiar with well-known techniques for preparation of oral formulations.

In certain embodiments, pharmaceutical composition includes at least about 0.1% by weight of the drug. The composition may include, for example, about 0.01% to about 0.1% by weight of the drug. In other embodiments, the pharmaceutical composition includes about 2% to about 75% by weight of the drug in the composition, or between about 25% to about 60% by weight of drug in the composition.

The pharmaceutical compositions set forth herein may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. The composition must be stable under the conditions of manufacture and storage, and preserved against the contaminating action of microorganisms, such as bacteria and fungi. It will be appreciated that exotoxin contamination should be kept minimally at a safe level, for example, less that 0.5 ng/mg protein.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. In many cases, it will be preferable to include isotonic agents, such as, for example, sugars, sodium chloride or combinations thereof.

In other embodiments, one may use nasal solutions or sprays, aerosols or inhalants in the present invention. Nasal solutions are usually aqueous solutions designed to be administered to the nasal passages in drops or sprays.

Sterile injectable solutions are prepared by incorporating the nanoparticles in the required amount in the appropriate solvent with various of the other ingredients enumerated above, as required, followed by sterilization.

2. Routes of Administration

Upon formulation, nanoparticles will be administered in a manner compatible with the dosage formulation and in such amount as is therapeutically effective. The nanoparticles can be administered to the subject using any method known to those of ordinary skill in the art. For example, a pharmaceutically effective amount of a composition comprising nanoparticles may be administered intravenously, intracerebrally, intracranially, intrathecally, into the substantia nigra or the region of the substantia nigra, intradermally, intraarterially, intraperitoneally, intralesionally, intratracheally, intranasally, topically, intramuscularly, intraperitoneally, subcutaneously, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (Remington's, 1990). In particular embodiments, the composition is administered to a subject using a drug delivery device.

3. Dosage

A pharmaceutically effective amount of the nanoparticles is determined based on the intended goal, for example inhibition of cell death. The quantity to be administered, both according to number of treatments and dose, depends on the subject to be treated, the state of the subject, the protection desired, and the route of administration. Precise amounts of the therapeutic agent also depend on the judgment of the practitioner and are peculiar to each individual.

For example, a dose of the drug may be about 0.0001 milligrams to about 1.0 milligrams, or about 0.001 milligrams to about 0.1 milligrams, or about 0.1 milligrams to about 1.0 milligrams, or even about 10 milligrams per dose or so. Multiple doses can also be administered. In some embodiments, a dose is at least about 0.0001 milligrams. In further embodiments, a dose is at least about 0.001 milligrams. In still further embodiments, a dose is at least 0.01 milligrams. In still further embodiments, a dose is at least about 0.1 milligrams. In more particular embodiments, a dose may be at least 1.0 milligrams. In even more particular embodiments, a dose may be at least 10 milligrams. In further embodiments, a dose is at least 100 milligrams or higher.

In other non-limiting examples, a dose of a drug may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges.

In certain embodiments, it may be desirable to provide a continuous supply of a pharmaceutical composition to the patient. This could be accomplished by catheterization, followed by continuous administration of the therapeutic agent. The administration could be intra-operative or post-operative.

### H. Combination Treatments

Certain embodiments of the present invention provide for the administration or application of one or more secondary forms of therapies for the treatment or prevention of a disease. For example, the disease may be a hyperproliferative disease, such as cancer.

The secondary form of therapy may be administration of one or more secondary pharmacological agents that can be applied in the treatment or prevention of cancer. If the secondary therapy is a pharmacological agent, it may be administered prior to, concurrently, or following administration of the nanoparticles.

The interval between the administration of the nanoparticles and the secondary therapy may be any interval as determined by those of ordinary skill in the art. For example, the interval may be minutes to weeks. In embodiments where the agents are separately administered, one would generally ensure that a significant period of time did not expire between the time of each delivery, such that each therapeutic agent would still be able to exert an advantageously combined effect on the subject. For example, the interval between therapeutic agents may be about 12 h to about 24 h of each other and, more preferably, within about 6 hours to about 12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations. In some embodiments, the timing of administration of a secondary therapeutic agent is determined based on the response of the subject to the nanoparticles.

Various combinations may be employed. For the example below an inhibitor of gene expression therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a standard therapy will include chemotherapy, radiotherapy, immunotherapy, surgical therapy or gene therapy and may be employed in combination with the inhibitor of gene expression therapy, anticancer therapy, or both the inhibitor of gene expression therapy and the anti-cancer therapy, as described herein.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1l and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor; vaccines such as gene therapy vaccines and pharmaceutically acceptable salts, acids or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gen silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p9′7), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### J. Kits and Diagnostic Agents

In various aspects of the invention, a kit is envisioned containing nanoparticles or ingredients for the formation of nanoparticles of the present invention in one or more suitable container means. A suitable container means is a container that will not react with components of the kit, such as an eppendorf tube, an assay plate, a syringe, a bottle, or a tube. The container may be made from sterilizable materials such as plastic or glass. In some embodiments, the kit includes a composition comprising nanoparticles in one or more container means. In other embodiments, the kit includes a single container means that comprises an SF polypeptide or a solution comprising an SF polypeptide, and a separate container means that comprises a drug.

In some further embodiments, the kit includes one or more therapeutic or diagnostic agents. The one or more therapeutic or diagnostic agents may be in the same container means with the polyphosphate and/or chitosan. The kit may further comprise a lipid in a suitable container means.

The kit may further include an instruction sheet that outlines the procedural steps of the methods, and will follow substantially the same procedures as described herein or are known to those of ordinary skill.

### Example 1

### Preparation and Characterization of Silk Fibroin-Chitosan Derived Curcumin Nanoparticles

### Materials and Methods

Preparation of Silk-Fibroin and Chitosan Blends. Raw Silk (Sao Paulo, Brazil) was generously donated by Dr. Sam Hudson (North Carolina State University, Raleigh, N.C.) and high molecular weight chitosan (82.7% deacetylation) was obtained from Sigma-Aldrich (St. Louis, Mo.). Processing of SF and preparation of silk fibroin-chitosan (SFCS) blends has been described previously (Gobin et al., 2005). In brief, raw silk was degummed in 0.25% (w/v) sodium carbonate and 0.25% (w/v) sodium dodecylsulfate (Sigma-Aldrich) for 1 hour at 100° C. and then dissolved in calcium nitrate tetrahydrate and methanol solution (molar ratio of 1:4:2 Ca:H₂O:MeOH) at 65° C. Chitosan was dissolved separately in 2% acetic acid solution at the same concentration as silk fibroin solution and was mixed together to prepare a particular blend. For example, three parts of SF were mixed with one part of CS to prepare 75:25 SFCS. The SFCS solution was then dialyzed against ultra-pure water for 4 days and filtered. The final solution was 10% (w/v) of SF or SFCS and diluted 100 times to make 0.1% solutions.

Preparation of nanoparticles. Curcumin powder was weighed (1 mg) and suspended in 100 μl of 25:75, 50:50 or 75:25 SFCS and SF only. These nanoparticles were prepared using the capillary microdot technique. The drug suspension was dispensed on glass slides via a microcapillary. The slides were then frozen overnight and lyophilized. Dry dots containing SFCS coated curcumin nanoparticles were scraped off the slides and collected in a 1.5 ml centrifuge tube. The SFCS coating was crystallized by suspending nanoparticles in 0.5 ml of 50:50 methanol and 1N sodium hydroxide solution (for SF coating, only methanol was used) for 15 minutes (Gobin et al., 2006). The suspension was centrifuged at 10,000 rpm for 10 minutes and supernatant removed. The pellet containing nanoparticles was rinsed with PBS twice to remove remaining methanol and sodium hydroxide. PBS rinses were performed by adding 0.5 ml of PBS to the nanoparticles and centrifuging at 10,000 rpm for 10 minutes. After the second rinse, the SFCS coated curcumin nanoparticles were suspended in PBS for further analysis.

Size measurement using Transmission electron microscopy (TEM). SFCS coated curcumin samples suspended in PBS were imaged using a JEM 1010 transmission electron microscope (TEM; JEOL USA Inc., Peabody, Mass.). Samples were placed on formver-coated and carbon-coated, copper grids treated with poly-L-lysine for 1 hour. The samples were then blotted dry and imaged. The size of the nanoparticles from TEM images was measured using ImageJ software.

Curcumin entrapment efficiency and release. After collecting the nanoparticles from glass slides, the methanol/sodium hydroxide rinse and 2 subsequent PBS rinses (as defined above) were collected to calculate entrapment efficiency. The curcumin that was not entrapped (not coated with SFCS) was completely soluble in organic solvent and hence was collected in methanol rinses. The amount of curcumin in the samples was measured by reading the absorbance at 450 nm using UV spectrophotometer (ThermoSpectronics, Rochester, N.Y.) and calculated from curcumin standard in ethanol (Bisht et al., 2007). Similarly, for drug release, the nanoparticle suspension was centrifuged at 10,000 rpm for 10 minutes and absorbance was measured in the supernatant. The pellet was again suspended in PBS for the next time point and kept at 37° C. shaker.

Cell Culture. Breast cancer cell lines MCF-7 (Her2−) and MDA-MB-453 (Her2+) were obtained from American Type Culture Collection (ATCC, Manassas, Va.). MCF-7 cells were cultured in Dulbecco Modified Eagles Medium with F-12 (Invitrogen, Grand Island, N.Y.) and MDA-MB-453 cells in Leibovitz's L-15 medium (ATCC). Both culture mediums were supplemented with 10% fetal bovine serum (Atlanta Biologicals, Lawrenceville, Ga.) and 1% antibiotics (Invitrogen).

Assay for intracellular uptake of curcumin. MCF-7 and MDA-MB 453 cells were seeded in 96-well plates (2000 cells/well) and incubated overnight. SFCS or SF coated nanocurcumin was added to each well at a concentration of 83.3 μg curcumin/well and incubated for 4 days. The nanoparticles containing medium were removed from the wells. Cells were lysed in 100 μl of dimethyl sulfoxide (DMSO, Fisher Scientific, Pittsburg, Pa.). The 50 μl cell lysis suspension was reserved for absorbance measurement and the other 50 μl for fluorescence measurements using a VersaFluor fluorometer (Bio-Rad Laboratories, Hercules, Calif.). Two filters were utilized with 480 nm excitation and 520 nm emission wavelengths. Curcumin has auto-fluorescence properties (Kunwar et al., 2006), therefore, fluorescence assays were used in conjunction with absorbance to confirm the curcumin uptake measurements.

Cell viability assay. To measure the efficacy of nanocurcumin on MCF-7 and MDA-MB 453, the cells were seeded in 96-well plates (2000 cells/well) and incubated overnight. Nanocurcumin coated with SFCS or SF was added to each well at a concentration of 83.3 μg curcumin/well and incubated for 4 days. The nanoparticles containing medium were removed from the wells and a CellQuanti-MTT cell viability assay kit (BioAssay Systems, Hayward, Calif.) was used to determine the viability of cells remaining in each well. Briefly, 80 μl of culture medium and 15 μl of MTT reagent were added to each well and incubated for 4 hours. Then, MTT solubilization solution (100 μl) was added to each well and plates were placed on a shaker for 1 hour. The absorbance readings were taken at 570 nm using MRX Microplate Reader (Dynex Technologies, Guernsey, Channel Islands, Great Britain).

Statistical analysis. All data analysis was performed using SigmaStat statistical program. One-way analysis of variance (ANOVA) was used with post-hoc Tukey test for pair-wise comparisons. All data was represented as mean±SEM and level of significance was chosen as p<0.05.

### Results

Nanoparticle Size Measurement. SF or SFCS coated curcumin particles were imaged using TEM to characterize the size. The shape of the nanoparticles was mostly round and square. Size measurements of curcumin particles from TEM images show that all formulations created particles of size less than 100 nm except 0.1% 50:50 SFCS (130±4.2 nm) as shown in FIG. 2. Particle size of curcumin coated with 0.1% SF was significantly lower than 0.1% 50:50 SFCS (p<0.001) and higher than 0.1% 25:75 SFCS and 0.1% 75:25 SFCS (p<0.001) coated particles. The particle size for 10% 75:25 SFCS was significantly higher than 10% SF and 10% 25:75 and 10% 50:50 SFCS (p<0.001).

Although there are differences in the size of the nanoparticles between high and low concentrations of the SF content, no specific trend was noted. The size of particles coated with 0.1% SF and 0.1% 50:50 SFCS was significantly higher than 10% SF and 10% 50:50 SFCS (p<0.001). However, 0.1% 25:75 SFCS coated nanoparticles were smaller than 10% 25:75 SFCS coated particles (p<0.05). The size of SF and SFCS control particles (without drug) were also measured and found to be comparable with curcumin loaded particles.

Curcumin Entrapment Efficiency. The entrapment of curcumin was more than 96% for SF coated nanoparticles for both 0.1% and 10% SF. The entrapment efficacy decreased to 64-73% for SFCS coated nanoparticles regardless of concentration of SF and CS (FIG. 2). Curcumin entrapment was significantly higher for 0.1% SF and 10% SF compared to all SFCS blends.

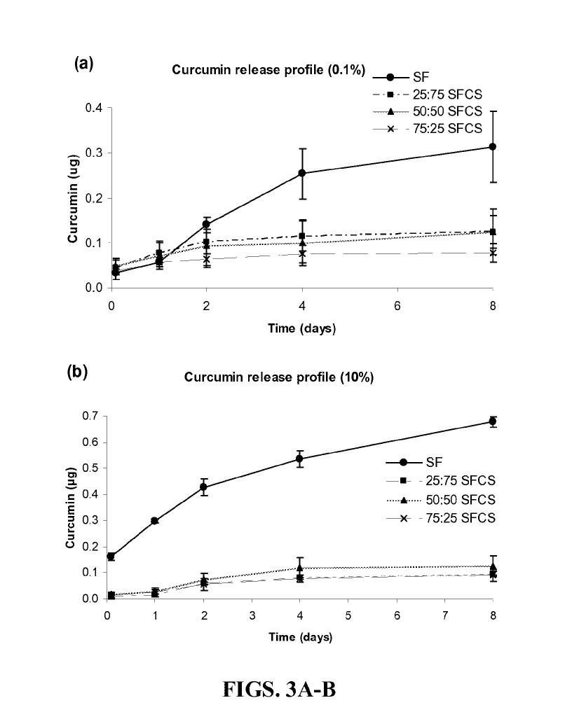

In Vitro Curcumin Release from Nanoparticles. The curcumin release profiles for both 0.1% and 10% coatings showed an initial burst release (up to 2 days) of curcumin from both SF and SFCS blends (FIG. 3). However, curcumin release from SFCS blends did not further increase over 8 days. But SF coated curcumin nanoparticles consistently released curcumin in large amounts over 8 days. As shown in FIG. 3, cumulative release of curcumin from SF coated nanoparticles at day 8 was significantly higher than all other SFCS blend nanoparticles (p<0.05 for 0.1% and p<0.001 for 10% solutions). Also, at day 8 the release from 10% SF was significantly greater than 0.1% SF (p<0.001).

The release of drugs from polymers has been previously described by the power law equation (Siepmann and Peppas, 2001).

$$\frac{{M}_{t}}{{M}_{∞}} = {kt}^{n}$$

Mₜ and M_{∞} are the amounts of drug at any time t and at infinite time, respectively. k and t are the constants and exponent dependent upon the composition/structure of the coating and release mechanism. Table 1 showed the values of n and k for all the nanoparticle formulations. For n<0.5, the drug release mechanism is diffusion based (Seipmann and Peppas, 2001); hence the curcumin release in all the formulations is diffusion based only. Based on k values, the rate of drug release was significantly higher for 10% SF as compared to 0.1% SF and all other SFCS blends (p<0.001).

TABLE 1
The exponent ‘n’ and constant ‘k’ values from the power
law equation for various nanoparticle formulations.

| Coatings | n | k (×10⁻⁴ per/day) |
|---|---|---|
| 0.1% SF | 0.55 ± 0.10 | 0.9 ± 0.1 |
| 0.1% 25:75 SFCS | 0.25 ± 0.05 | 1.0 ± 0.4 |
| 0.1% 50:50 SFCS | 0.17 ± 0.05 | 1.0 ± 0.5 |
| 0.1% 75:25 SFCS | 0.15 ± 0.05 | 0.8 ± 0.1 |
| 10% SF | 0.32 ± 0.03 | 3.0 ± 0.0 |
| 10% 25:75 SFCS | 0.28 ± 0.18 | 0.5 ± 0.07 |
| 10% 50:50 SFCS | 0.48 ± 0.17 | 0.6 ± 0.06 |
| 10% 75:25 SFCS | 0.45 ± 0.08 | 0.6 ± 0.2 |

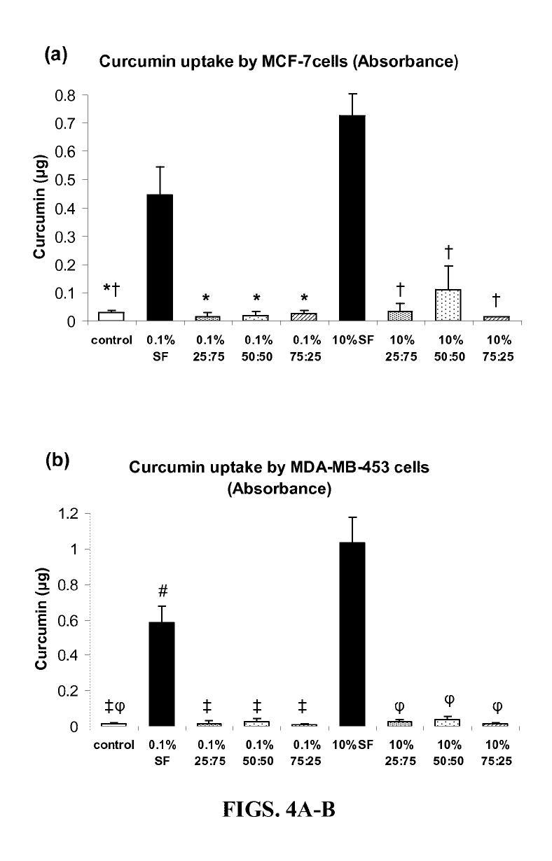

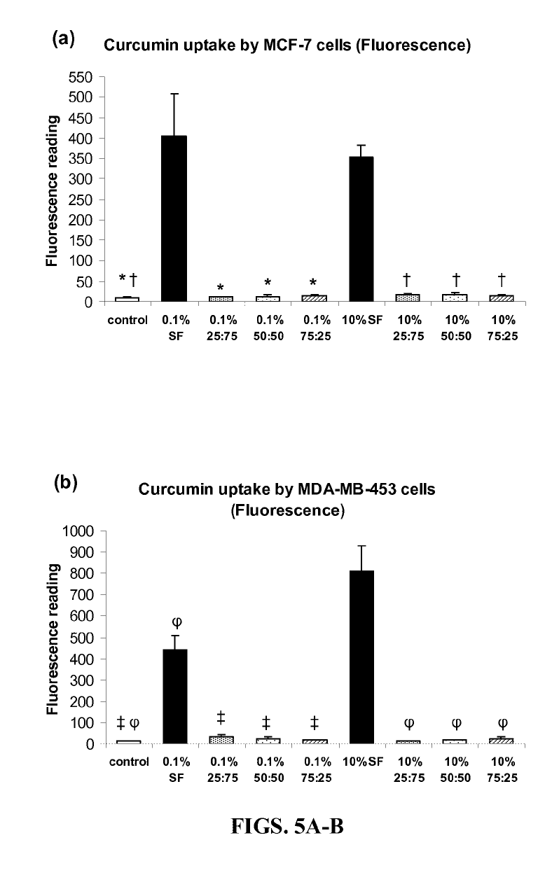

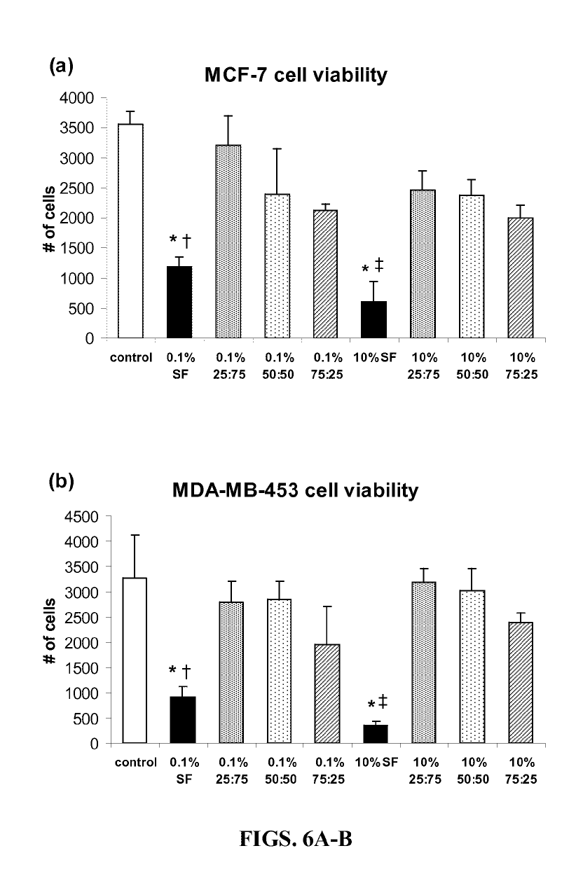

Intracellular Uptake of Curcumin. The absorbance measurement of intracellular uptake of curcumin show that the curcumin uptake was highest from SF coated nanoparticles as compared to all other SFCS blends (FIG. 4). These differences were similar for both MCF-7 and MDA-MB-453 cells. The fluorescence measurements also showed similar uptake data as compared to the absorbance measurements (FIG. 5). Interestingly, curcumin uptake by MDA-MB-453 cells was higher from 10% SF coated nanoparticles as compared to 0.1% SF coated nanoparticles as measured by both absorbance and fluorescence.

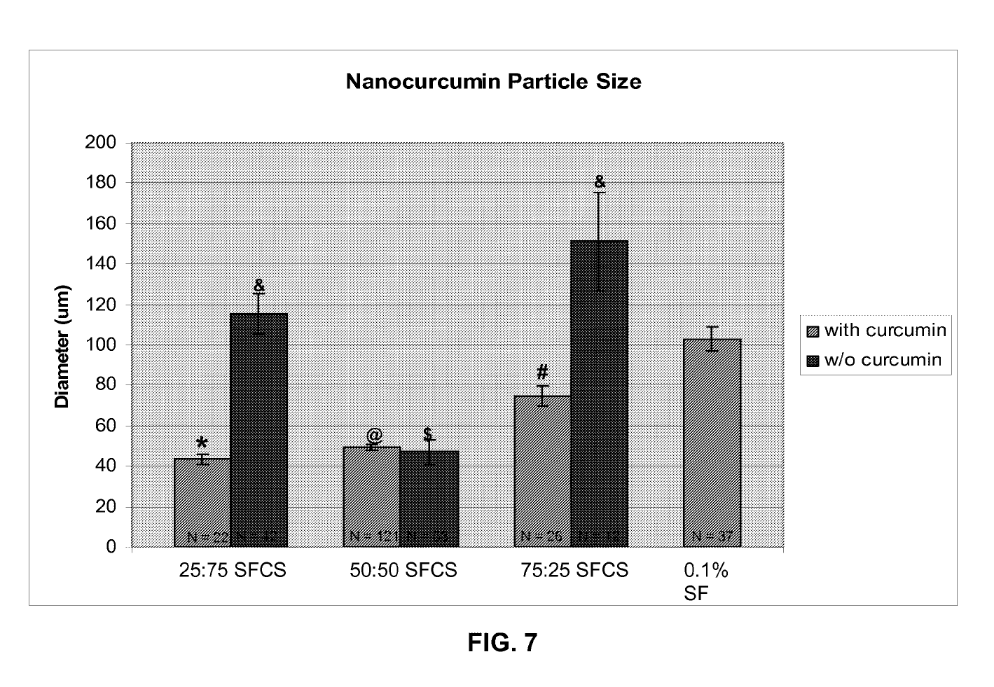

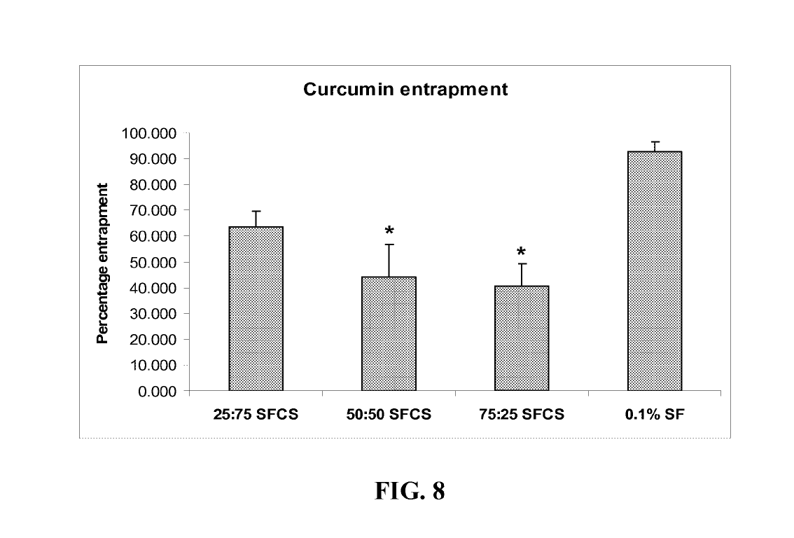

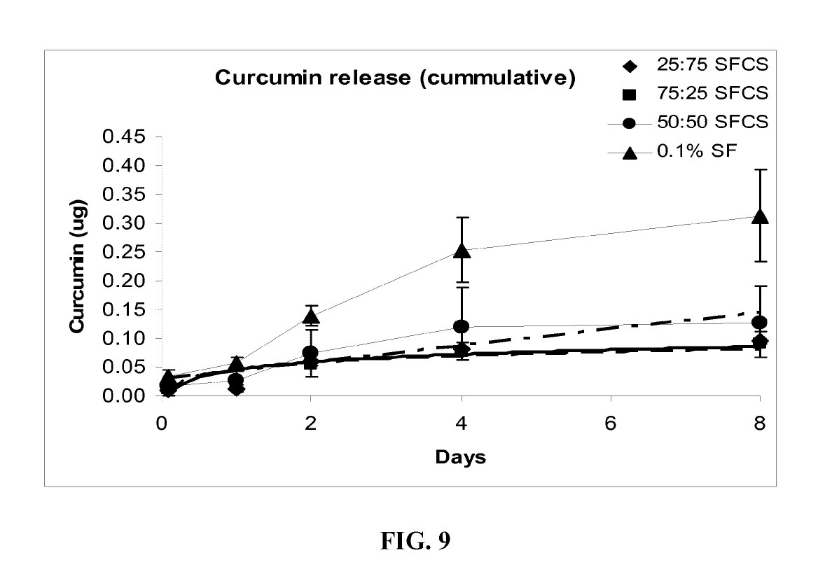

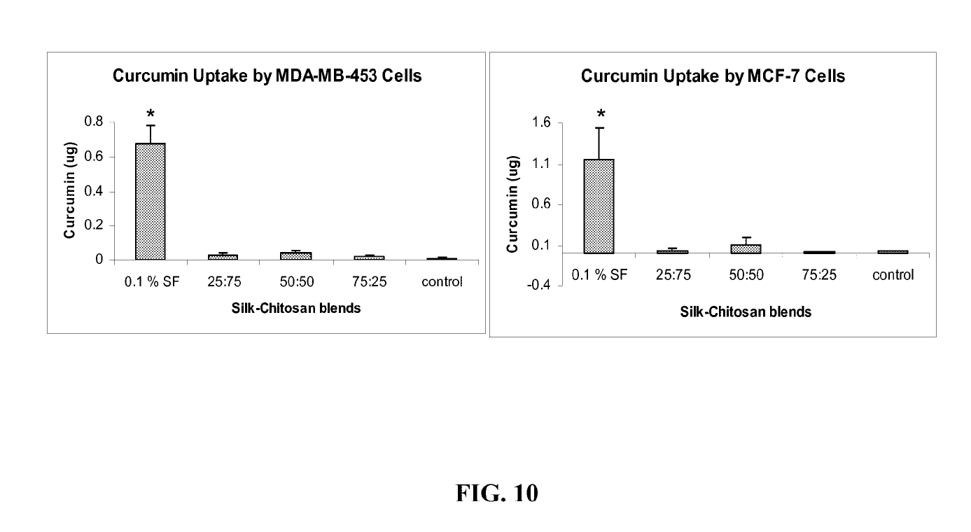

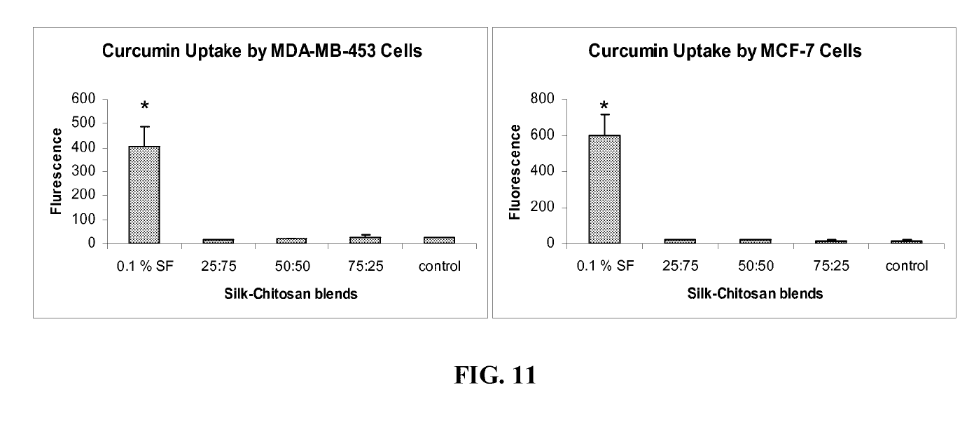

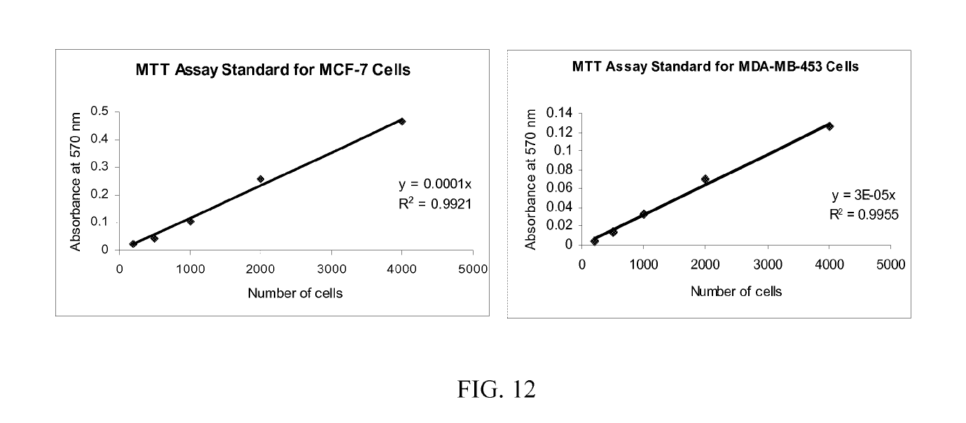

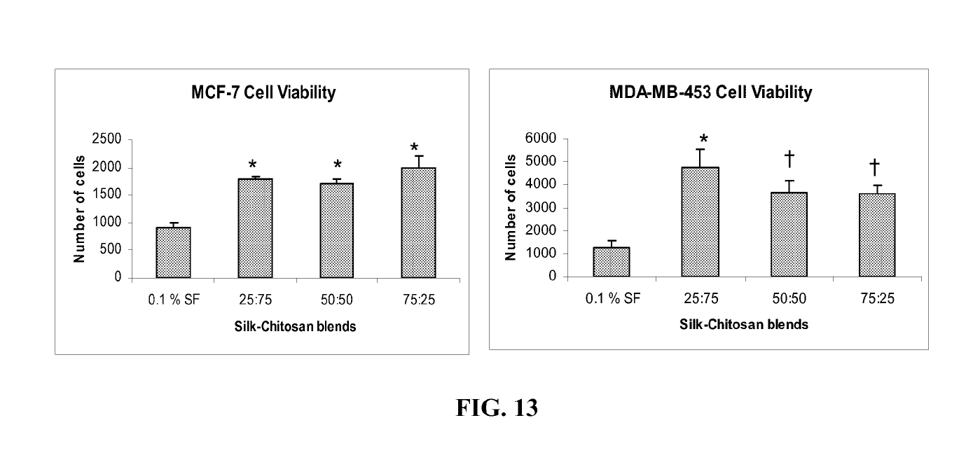

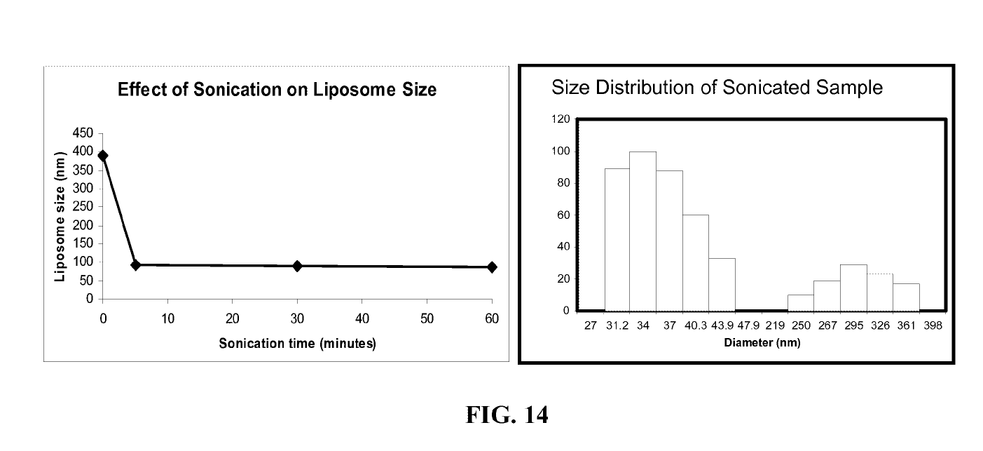

Curcumin Nanoparticle Efficacy on Breast Cancer Cells. The efficacy of curcumin nanoparticle formulations was measured on both MCF-7 and MDA-MB-453 cells using MTT assay as shown in FIG. 7. The number of cells in control samples (no nanoparticles) increased from 2000 (initial density) to 3563±215 (MCF-7) and 3267±864 (MDA-MB-453) over a period of 4 days. Exposure of 0.1% SF and 10% SF nanocurcumin to MCF-7 and MDA-MB-453 cells significantly decreased number of viable cells compared to controls (p<0.01). Also, the decrease in cell viability with 0.1% SF was significantly different than 0.1% 25:75 SFCS for MCF-7 cells (p<0.01) and 0.1% 50:50 SFCS for MDA-MB-453 cells (p<0.01). Similarly, 10% SF nanocurcumin had significantly higher efficacy against both types of breast cancer cells as compared to 10% 25:75 SFCS and 10% 50:50 SFCS (p<0.05).

### Example 2

### Preparation and Characterization of Emodin-Loaded Liposomes for Embedding Within Silk Fibroin/Chitosan

The objective of this study was to prepare nanoparticles for embedding within silk fibroin/chitosan-tissue flap composite used to fill defects after tumor resection in cancer patients. Emodin loaded liposomes were prepared, and these liposomes were entrapped within an SFCS composite. Liposome size release and drug release were examined.

Emodin is a selective receptor tyrosine kinase inhibitor. It is effective for HER-2/neu over expressing breast cancer cells. It functions to sensitize cells to other chemotherapeutic agents.

Liposomes are phospholipid bilayer microscopic vesicles. Drugs may be incorporated within the lipid bilayer or in the hydrophilic core. Liposomes are biodegradable, have high tissue targeting, lack immunogenicity and have low toxicity.

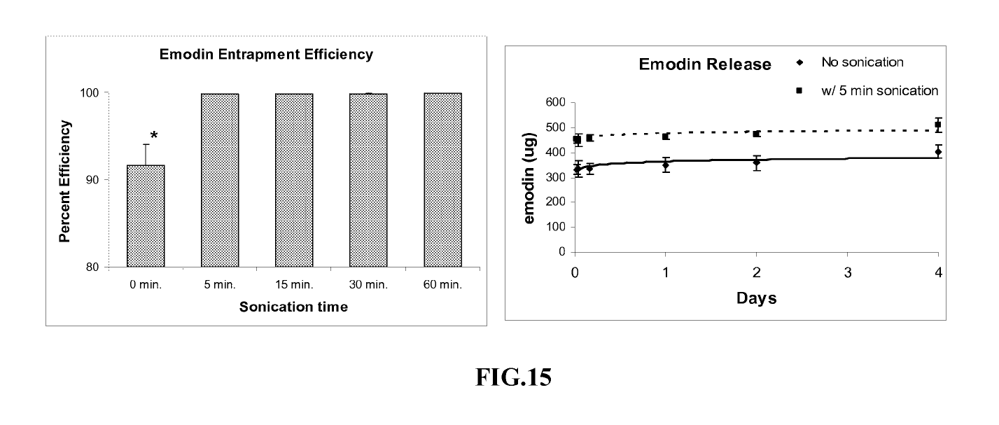

Emodin loaded liposomes were sonicated at different times (0 min, 5 min, 15 min, 30 min, and 60 min) to reduce particle size. Sonication was found to be effective for reducing liposome size (FIG. 14). Liposome size was found to be less than 100 nm after 5 minutes of sonication. There was found to be a higher emodin entrapment and release with sonication (FIG. 15). Entrapment efficiency was significantly lower without sonication (*p<0.01 vs. all sonication time points). A higher drug release was also associated with sonication (FIG. 15).

All of the nanoparticles, composition, and methods disclosed and claimed herein can be made and executed without undue experimentation in light of the present disclosure. While the nanoparticles, compositions, and methods of this invention have been described in terms of preferred embodiments, it will be apparent to those of skill in the art that variations may be applied to the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,282,351
U.S. Pat. No. 4,806,355
U.S. Pat. No. 4,806,474
U.S. Pat. No. 4,870,287
U.S. Pat. No. 5,015,476
U.S. Pat. No. 5,093,489
U.S. Pat. No. 5,263,992
U.S. Pat. No. 5,270,419
U.S. Pat. No. 5,576,881
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,846,945
U.S. Pat. No. 5,902,800
U.S. Pat. No. 6,127,143
U.S. Pat. No. 6,245,537
U.S. Pat. No. 6,267,776
U.S. Pat. No. 6,302,848
U.S. Pat. No. 6,310,188
U.S. Pat. No. 6,325,810
U.S. Pat. No. 6,337,198
U.S. Pat. No. 6,372,244
U.S. Pat. No. 6,379,690
U.S. Pat. No. 6,387,413
U.S. Pat. No. 6,395,734
U.S. Patent Pubin. 20030129730
U.S. Patent Pubin. 20030165548
U.S. Patent Pubin. 20040247632
U.S. Patent Pubin. 20050042735
U.S. Patent Pubin. 20050226938
U.S. Patent Pubin. 20050245482
U.S. Patent Pubin. 20050260706
U.S. Patent Pubin. 20060019348
U.S. Patent Pubin. 20060094666
U.S. Patent Pubin. 20060189573
U.S. Patent Pubin. 20060277632
U.S. Patent Pubin. 20070116767
U.S. Patent Pubin. 20070167400
U.S. Patent Pubin. 20070187862
U.S. Patent Pubin. 20070311468
U.S. Patent Pubin. 20080176960
Allan and Peyron, Carbohydr Res., 277(2):257-272, 1995.
Altman et al., Biomaterials, 24:401-16, 2003.
Anand et al., Mol. Pharm., 4:807-18, 2007.
Austin-Ward and Villaseca, Revista Medica de Chile, 126(7):838-845, 1998.
Bayomi et al., Pharm. Acta Helv., 73(4):187-192, 1998.
Bisht et al., J. Nanobiotechnology, 5:3, 2007.
Bukowski et al., Clinical Cancer Res., 4(10):2337-2347, 1998.
Cheema et al., Int. J. Pharm., 341:221-9, 2007.
Christodoulides et al., Microbiology, 144(Pt 11):3027-3037, 1998.
Davidson et al., J. Immunother., 21(5):389-398, 1998.
Domard and Cartier, Int. J. Biol. Macromol., 11(5):297-302, 1989.
Genta et al., Drug Dev. Ind. Pharm., 24(8):779-784, 1998.
Gobin et al. Int. J. Nanomedicine, 1:81-7, 2006.
Gobin et al., J. Biomed. Mater. Res., 74: 465-73, 2005.
Gobin et al., Tissue Eng., 12:3383-94, 2006.
Gupta and Ravi Kumar, J. Mater Sci. Mater Med., 12(9):753-759, 2001.
Hanibuchi et al., Int. J. Cancer, 78(4):480-485, 1998.
Hellstrand et al., Acta Oncologica, 37(4):347-353, 1998.
Hong et al., Clin. Cancer Res., 5:1884-91, 1999.
Hui and Hashimoto, Infection Immun., 66(11):5329-5336, 1998.
Ju et al., Gene Ther., 7(19):1672-1679, 2000.
Katas and Alpar, J. Control Release, 115(2):216-225, 2006.
Ko et al., J Microencapsul., 20(6):791-797, 2003.
Kunnumakkara et al., Cancer Lett., 269:199-225, 2008.
Kunwar et al., Biochim. Biophys. Acta, 1760:1513-20, 2006.
Li et al. Proteins, 44(2):119-22, 2001.
Li et al., Cancer, 104:1322-31, 2005.
Liu et al., Nan Fang Yi Ke Da Xue Xue Bao., 27(2):184-7, 2007.
McCutcheon's Detergents and Emulsifiers, 1998.
McCutcheon's Functional Materials McCutcheon Publishing Co., New Jersey, 1998.
Mi et al., Advanced Drug Deliv. Rev., 52(1):145-150, 1999.
Mitchell et al., Ann. NY Acad. Sci., 690:153-166, 1993.
Mitchell et al., J. Clin. Oncol., 8(5):856-869, 1990.
Nazarov et al. Biomacromolecules, 5(3):718-26, 2004.
Pietras et al., Oncogene, 17(17):2235-2249, 1998.
Qin et al., Proc. Natl. Acad. Sci. USA, 95(24):14411-14416, 1998.
Ravindranath and Morton, Intern. Rev. Immunol., 7: 303-329, 1991.
Remington's Pharmaceutical Sciences, 18th Ed. Mack Printing Company, 1289-1329, 1990.
Roden et al., Fed. Proc., 44:373-80, 1985.
Rosenberg et al., Ann. Surg. 210(4):474-548, 1989.
Rosenberg et al., N. Engl. J. Med., 319:1676, 1988.
Sahu et al., Acta Biomater., 2008.
Santin et al., J. Biomed. Mater. Res., 46:382-9, 1999.
Siepmann and Peppas, Adv. Drug Deliv. Rev., 48:139-57, 2001.
Sou et al., Int. J. Pharm., 352:287-93, 2008.
Tiyaboonchai et al., Int. J. Pharm., 337:299-306, 2007.
VandeVord et al., J. Biomed. Mater. Res., 59:585-90, 2002.
Varum et al., Carbohydr. Res., 211(1):17-23, 1991.
Wang et al. J. Control Release, 121:190-9, 2007.
Wang et al., J. Control Release, 117:360-70, 2007.
Wenk et al., J. Control Release 2008.

1. A nanoparticle for delivery of a drug and/or nutraceutical, comprising:
(a) a silk fibroin (SF) polypeptide;
(b) a chitosan; and
(b) a drug and/or nutraceutical,
wherein the nanoparticle has a diameter of about 1 nm to about 500 nm.

2. The nanoparticle of claim 1, wherein the nanoparticle has a diameter of about 1 nm to about 300 nm.

3. The nanoparticle of claim 2, wherein the nanoparticle has a diameter of about 1 nm to about 100 nm.

4. The nanoparticle of claim 1, wherein a composition comprising the SF coats a core that comprises a drug.

5. The nanoparticle of claim 1, wherein the drug is a small molecule, a peptide, a polypeptide, a protein, an antibody, an antibody fragment, a DNA or a RNA.

6. The nanoparticle of claim 1, wherein the drug is a therapeutic agent.

7. The nanoparticle of claim 6, wherein the therapeutic agent is an anticancer agent, an anti-inflammatory agent, an antimicrobial agent, an analgesic, a hormone, or an anesthetic agent.

8. The nanoparticle of claim 7, wherein the therapeutic agent is an anticancer agent.

9. The nanoparticle of claim 8, wherein the anticancer agent is curcumin, emodin, thiotepa, cyclosphosphamide, busulfan, topotecan, chlorambucil, melphalan, carmustine, lomustine, actinomycin, bleomycin, dactinomycin, daunorubicin, mitomycin C, methotrexate, 5-fluorouracil (5-FU), 6-mercaptopurine, thioguanine, arabinoside, cyclophosphamide, thiotepa, paclitaxel, doxetaxel, chlorambucil, gemcitabine, 6-thioguanine, mercaptopurine, methotrexate, cisplatin, oxaliplatin, carboplatin, vinblastine, etoposide, vincristine, retinoic acid, cisplatin (CDDP), carboplatin, procarbazine, or mechlorethamine.

10. The nanoparticle of claim 6, wherein the drug is a curcuminoid.

11. The nanoparticle of claim 9, wherein the drug is emodin.

12. The nanoparticle of claim 1, wherein the drug is a diagnostic agent.

13. The nanoparticle of claim 1, wherein the chitosan has a deacetylation degree of greater than about 70%.

14. The nanoparticle of claim 13, wherein the chitosan has a deacetylation degree of greater than about 80%.

15. The nanoparticle of claim 1, wherein the weight ratio of the SF polypeptide to the chitosan is about 2 to about 4.

16. The nanoparticle of claim 1, wherein the nanoparticle comprises two or more drugs.

17. The nanoparticle of claim 1, wherein the nutraceutical is a vitamin or dietary supplement.

18. The nanoparticle of claim 17, wherein the vitamin or dietary supplement is vitamin A, vitamin D, acetyl carnitine, vitamin B-12, vitamin B-3, vitamin B-6, C ester, calcium citrate, cholesterol, chromium, CLA (tonalin), cod liver oil, creatine, vitamin D, CHEA, Dong Quai, vitamin E, fish oil, gaba, vitamin A, acidophilus, alpha lipoic, vitamin B-1, vitamin B-150, vitamin B-5, vitamin B-9, biotin, vitamin C, calcium, chlorella, choline, coenzyme Q-10, cranberry extract, D-ribose, EDTA, flaxseed oil, acai, arginine, aspirin, vitamin B-100, vitamin B-2, B-complex, beta-carotene, brewers yeast, magnesium, chia, chlorophyll, chondroitin, glucosamine, DHA, DMAE, ester-C, fiber, folic acid, vitamin B-9, ginseng, gymneme sylvestre, hawthorn, hoodia, vitamin K, L-carnitine, hyaluronic acid, inositol, L-lysine, lecithin, lipoic, melatonin, glutamine, niacin, pantothenic acid, peppermint extract, phosphotidyl serine, potassium, policosanol, psyllium, pycnogenol, quercetin, resveratrol, retinol, shark cartilage, sambucus, selenium, silica, ubiquinol, valerian, vitamin B-15, chasteberry extract, wild yam extract, whey protein, yeast, or zinc.

19. The nanoparticle of claim 1, wherein the nutraceutical is an herb.

20. The nanoparticle of claim 19, wherein the herb is buta, aligator yam, alfalfa, almond, amalaki, ashwagandha, asoka tree, ambrette plant, apricot, arecanut palm, arjuna, aloe vera, arnica, ash gourd, ashoka tree, asparagus, babchi seed, bacopa, bael tree, bahama grass, banyan tree, barbados aloe, buddhist bauhinia, belliric myrobalan, bengal gram, bermuda grass, betelnut palm, bindweed, bishop's weed, billilotan, bitter melon, black nightshade, boerhavia, bitter gourd, black cumin, black nightshade, black-oil plant, black pepper, black plum, bonduc fruit, boswellia, bread wheat, butea gum tree, cinnamon, cajuput tree, calendula, cal mint, camphor, caper, cayenne, cal mint, chyavanprash, clove, country mallow, crowfoot, caraway, cinnamon, coconut extract, cohost, cardamom, carilla fruit, carum, cassia, castor, celery, centella, ceylon cinnamon, chebulic myrobalan, chickpea, chicory, chilli, chiretta, chir pine, citron, climbing staff tree, clove, coconut, coffee-senna, common mallow, common olive, common sesban, common wheat, coneru, connessi bark, coomb teak, coriander, corn poppy, costus, country mallow, cow-itch plant, cowhage, crab apple, crowfoot, cucumber, cultivated apple, cultivated carrot, cumin, curacao aloe, cutch tree, date, dead sea apple, dandelion extract, deodar, dhub grass, downy oak, drumstick, didymocarpus, evening primrose oil, East Indian globe thistle, elderberry extract, Egyptian rattle pod, elephant creeper, emblic myrogalan, English garden marigold, eucalyptus, false black pepper, false pepper, fennel, fenugreek, fenugreek, fire flame bush, five-leaved chaste tree, elderberry extract, foetid cassia, gall nut, galls, garcinia, garden rue, garlic, giant potato, giant reed, gingelly, ginger, golden dock, golden shower, gotu kola, grape, greater galangal, gulancha tinospora, guduchi, guggul, gymnema, haritaki, henbane, Himalayan cedar, Himalayan silver birch, holy basil, horsegram, horse radish, hydrophilia, hyssop, aloe, berberry, bdellium, beech, cotton plant, dill, gooseberry, gum-arabic tree, jojoba, kelp, krill oil, jeevanti, king bitters, kino tree, laburnum, lilac, long pepper, madder, malabar nut, maca, nattokinase, goji, mango, morning glory, mucuna, musk mallow, musk root, nard, neem, nut grass, nutmeg, paper birch, pennywort, pasanavheda, pellitory, pitasara, pomegranate, prickly chaff flower, sarsaparilla, spikenard, sweet fennel, valerian, winter green, ink nut, intellect tree, ironwood tree, jafarabad aloe, jaman, jambolan, java plum, khas-khas, khus-khus, land caltrops, cardamum, lemon, lentil, lettuce, licorice, liquorice, lodh tree, loose skinned orange, loose jackot orange, lovage, malabar nut, mango, mangosteen, morning glory, grape seed extract, grape extract, green tea extract, lavender, lutein, milk thistle, nettle, mucuna, musk mallow, musk root, neti pot, noni, neem, negro coffee, nut grass, nutmeg, orchid tree, pasanavheda, pellitory, pitasara, papaya, peppermint, Persian rose, pistachio, pinang palm, pomegranate, pongam oil tree, pot-marigold, prickly-chaff flower, puncture vine, purging cassia, purple fleabane, rauwolfia, red pepper, red poppy, rough chaff tree, rohida tree, rosemary, rubbish cassia, sacred basil, saffron, salep orchid, sal tree, sandalwood tree, sarala, saxifraga ligulata, shavatari, shilajeet, shilapushpa, sins, small caltrops, spreading hogweed, sedge, sedom apple, sensitive plant, sesame, shoe-flower, shrivasa, silk cotton tree, small caltrops, small fennel, snake wood, soap nut, soapnut-tree of North India, sodom apple, Spanish jasmine, stevia, saw palmetto extract, spirulina, tea extract, turmeric, St. John's wort, Spanish Pellitory, spearmint, spreading hogweed, stinking weed, stone flower, strychnous tree, succory, sunflower, swallow-wort, sweet flag, sweet indrajao, sweet root, sweet violet, tamarisk, tankana, Tasmanian blue gum tree, tea treethistle extract, tonalin, tellicherry bark, teri pods, caper bush, the creat, thorn apple, three leaved caper, thyme-leaved gratiola, tinospora gulancha, tomato, triphala, toothache tree, touch me not, tree turmeric, umbrellas edge, velvet bean vibhitake, wild asparagus, yarrow, or yohimbe.

21. A drug delivery nanoparticle comprising:
(a) a silk fibroin (SF) polypeptide; and
(b) a curcuminoid,
wherein the nanoparticle has a diameter of about 1 nm to about 500 nm.

22. (canceled)

23. A nanoparticle comprising:
(a) a silk fibroin (SF) polypeptide; and
(b) R15K peptide,
wherein the nanoparticle has a diameter of about 1 nm to about 500 nm.

24. A nanoparticle comprising:
(a) a silk fibroin (SF) polypeptide; and
(b) emodin,
wherein the nanoparticle has a diameter of about 1 nm to about 500 nm.

25. A pharmaceutical and/or nutraceutical composition comprising a nanoparticle as set forth in claim 1 and a pharmaceutically acceptable carrier.

26. (canceled)

27. A method of delivering a drug and/or nutraceutical to a subject, comprising administering to the subject an effective amount of a composition comprising a nanoparticle, wherein the nanoparticle is a nanoparticle as set forth in claim 1.

28-39. (canceled)

40. A method of treating breast cancer in a subject, comprising administering to the subject a pharmaceutically effective amount of a composition comprising:
(a) a silk fibroin (SF) polypeptide; and
(b) an anticancer agent,
wherein the nanoparticle has a diameter of about 1 nm to about 500 nm.

41-43. (canceled)

44. A method of preparing a nanoparticle or delivery of a drug and/or nutraceutical, comprising the steps of:
(a) preparing a composition comprising a i) SF polypeptide, ii) a drug or nutraceutical, and iii) a carrier;
(b) dispensing droplets of the composition of (a) on a surface;
(c) lyophilizing the surface of (b) to remove the carrier, wherein dried dots comprising SF polypeptide and drug or nutraceutical are formed on the surface;
(d) removing the composition of (c) from the surface,
wherein the composition of (c) comprises nanoparticles having a diameter of about 1 nm to about 500 nm.

45-54. (canceled)

55. A kit comprising a first sealed container comprising a nanoparticle as set forth in claim 1.

56-58. (canceled)


SEQUENCE LISTING (6 sequences)

SEQ ID NO: 1 - protein, 262 aa, Bombyx mori
MKPIFLVLLVATSAYAAPSVTINQYSDNEIPRDIDDGKASSVISRAWDYVDDTDKSIAILNVQEILKDMASQGDYASQASAVAQTAGIIAHLSAGIPGDACAAANVINSYTDGVRSGNFAGFRQSLGPFFGHVGQNLNLINQLVINPGQLRYSVGPALGCAGGGRIYDFEAAWDAILASSDSGFLNEEYCIVKRLYNSRNSQSNNIAAYITAHLLPPVAQVFHQSAGSITDLLRGVGNGNDATGLVANAQRYIAQAASQVHV

SEQ ID NO: 2 - protein, 251 aa, Antipaluria urichi
SGSGSGAGSGSGAGSGSGAGSGSGAGSGSGAGSGSGAGSGAGSGSGAGSGSGAGSGSGAGSGSGAGSGSGSGSGSGSGSGAGSGSGSGAGSGAGSGSGAGSGSGSGSGSGAGSGSGSGAGSGSGAGSGSGSGAGSGSGSGSGSGSGSGSGSGAGSGAGSGAGSGSGAGSGSGSGSGAGSGSGSGSGSGSGSGVGSGAGSGSGSGSGSGSGSGSGSGGDNEADSGEIDCPVPADLYDSNDWDAIDAYVERYC

SEQ ID NO: 3 - protein, 443 aa, Oecophylla smaragdina
MKIPAILATSLFVWGLVGASELVGSDASATASAEASASSSAYGSKYGIGSGAVSGASASASASASASASASSAPAIEGVNVGTGVSNTASASAEALSRGLGIGQAAAEAQAAAAGQAAIAAKSCALAAKSTAQAVALVEKVARAEVDLAESARKATRLSAEAAKAAAEVEKDLVGLRGAAGKLNLAARAGSKAQERANEDSIEANELAQATAAAGAEAEAKANAAQEAGASALAIAQAALNIEQETVKLTRQAQNTRLRSENILAAASNARAIASAEAEASSDLNNRANAARSNARAAAETRAVATEAASTAEIAAYSSSEKGEITNPGPLPKIVSVTAGLTQNEIAGSGAAASASASALASASAGAGAGAGAGAGASAGAGAVAGAGAGAGAGASAGASAGANAGAGASSLLLPQSKLHPISRSSASASASAEAEANSSAYA

SEQ ID NO: 4 - protein, 400 aa, Oecophylla smaragdina
MKIPAIFVTSLLAWGLASGGVIGPDTSSSSQASASASASASASASSSASIGYNELHKSINAPALAVGVKNGGVDVAKGAAVVESAISDVSTLTDDRTLNGLAIIGNSAESLARAQASSSASAGAKANALIKQSIAAIEITEKAEYLASIVATKAAKAAEATAAATARATAVAEAAKVSSEQFAAEARAAADAEAKANAASIIANKANAVLAEAATGLSASAGKAQQSATRALQAARAAAKAQAELTQKAAQILVLIAEAKAAVSRASADQSVCTSQAQAASQIQSRASAAESAASAQSEANTIAAEAVARADAEAASQAQAWAESFKRELSSVVLEAEANASASASAGALASGSSSSGASSSADASAGASSYGSLGGYRHGGSFSEASAAASAASRAEAA

SEQ ID NO: 5 - protein, 441 aa, Myrmecia forficata
MKIPAILATSLLIWGLVGASELESEASAAASAQAEASSSGRSGKLSASQASASASASASAGSRGGSKGGWGQLRRGDVKSEAKSAAAIAVEGAKIGTGIGNTASASAEALSRGLGIGQAAAEAQAAAAGQAEVAAKSCELADKTTAKAVAMVEAAAEAEIEVANQEVAAVKLSTWAAKAARIVEEDSAAVRAAAGKLLLAARAAAAAERRANEESEAANELAQASSAAAAEAEAKANAGREAAAAALAIAEAAVAIEQEAVILARKAQDARLNAEAAAAAAMNARVIASAESEASEDLENRASVARASAAGAAEAKAIATDAGATAEIAAYSWAKKGELINPGPLPKIISVNADLSKSEVEAMKITRGQVQEVKKISTHKGGWGWGKEGRSKVSSNASARASASANAAAGSLGSKWGRQLSASSASADANAEADSQLLKVW

SEQ ID NO: 6 - protein, 357 aa, Bombus terrestris
MKIPSILAVSLLVWGLASAGKPLIANAQIGKVKTETSSSSEIETLVSGSQTLVAGSETLASESEALASKSEALTSEAEIASVTTKDELILKGEAITGKKLGTGASEVAAASGEAIATTLGAGQAAAEAQAAAAAQAKSAAAAAANAGESSNSAAALVAAAAAAQGKAAAAAAAATKASLEAADAAEEAESAVALARAASAKAEALASTAAAANTRAALQAEKSNELAQAEAAAAAEAQAKAAAAAKATQLALKVAETAVKTEADAAAAAVAAAKARAVADAAASRATAVNAIAEAEERDSAQAENTAGVAQAALAAAEAQDSCIGAAATPRHSSSYAWWKLRITSLIVILSPRNRRT